(19) United States
(12) Patent Application Publication
(10) Pub. No.: US 2024/0301141 A1
(43) Pub. Date: Sep. 12, 2024

(54) SILICONE POLYMER COMPOUND AND TRANSDERMAL DELIVERY SYSTEM COMPRISING SAME

(71) Applicant: UNIVERSITY-INDUSTRY COOPERATION GROUP OF KYUNG HEE UNIVERSITY, Yongin-si (KR)

(72) Inventors: Dokyoung KIM, Seoul (KR); Rae Hyung KANG, Namyangju-si (KR)

(73) Assignee: UNIVERSITY-INDUSTRY COOPERATION GROUP OF KYUNG HEE UNIVERSITY, Yongin-si (KR)

(21) Appl. No.: 18/654,778

(22) Filed: May 3, 2024

Related U.S. Application Data
(63) Continuation of application No. PCT/KR2022/006036, filed on Apr. 27, 2022.

(30) Foreign Application Priority Data
Nov. 3, 2021 (KR) 10-2021-0149450
Apr. 27, 2022 (KR) 10-2022-0051881

(51) Int. Cl. C08G 77/04; A61K 9/00; A61K 9/48; A61K 31/4709; A61K 31/4745; A61K 31/704; A61K 38/08; A61K 47/34; C08G 77/06

(52) U.S. Cl. CPC C08G 77/04; A61K 9/0014; A61K 9/4816; A61K 31/4709; A61K 31/4745; A61K 31/704; A61K 38/08; A61K 47/34; C08G 77/06

(57) ABSTRACT

The present invention relates to a silicone polymer compound, a method for preparing same, a composition for transdermal delivery comprising same, and an anti-cancer composition comprising same, wherein a cyclic silane is used as a monomer, and the molecular weight, viscosity, contact angle, moisture content, drug encapsulation ratio, and release behavior of a synthesized silicone polymer compound can be controlled by controlling polymerization conditions such as the type of initiator, ratio of initiator to monomer, and polymerization temperature, and the synthesized silicone polymer compound can thus be used as a transdermal delivery system loaded with a drug and various active substances. In addition, by encapsulating an anticancer agent in the silicone polymer compound of the present invention, the effect of the anticancer agent can be enhanced, and the silicone polymer compound can thus also be utilized for an anticancer use.

### CROSS-REFERENCE TO RELATED APPLICATIONS

Pursuant to 35 USC 120 and 365(c), this application is a continuation of International Application No. PCT/KR2022/006036 filed on Apr. 27, 2022, and claims the benefit under 35 USC 119(a) of Korean Application No. 10-2021-0149450 filed on Nov. 3, 2021 and Korean Application No. 10-2022-0051881 filed on Apr. 27, 2022, in the Korean Intellectual Property Office, the entire disclosures of which are incorporated herein by reference for all purposes.

### TECHNICAL FIELD

The present invention relates to a silicone polymer compound, a transdermal delivery system including the same, and an anti-cancer composition including the same.

### BACKGROUND ART

A drug delivery system (DDS) is a general term for technologies that optimize drug treatment by designing a formulation to efficiently deliver a required amount of drug in order to minimize the side effects of existing drugs and maximize their efficacy. The DDS may be classified into an oral type, an injection type, a pulmonary inhalation type, a transdermal type, a mucosal administration type, an insertion type, etc. depending on a delivery route, a type of drug, and a type of delivery technology.

Currently, a drug delivery system based on oral delivery is still the most preferred method for administering pharmaceutical ingredients due to their unique advantages, including various formulations, ease of painless administration, feasibility for solid formulations, self-administration, and patient compliance. Despite these advantages, an oral drug delivery system has several limitations, such as drug stability in the gastrointestinal tract, hepatic first-pass metabolism, low water solubility, and limited drug absorption by physiological barriers. For example, there is a possibility that the drug may be degraded by enzymatic biochemical reactions or acid hydrolysis in the stomach, and the solubility problem of the drug in intestinal fluid and the permeability of the drug through the intestinal membrane limit the rate of drug absorption, resulting in low bioavailability of the drug.

To overcome the shortcomings of DDS based on the aforementioned oral delivery, transdermal drug delivery has been proposed as a new method. A transdermal drug delivery system (TDDS) is a method of using the skin as a drug administration site, and has advantages of being painless and less invasive, avoiding a first-pass metabolism, ease of application and administration, requiring no specialized personnel, and reducing the frequency of administration. In addition, the TDDS is attracting attention from many researchers because the TDDS may be used to deliver various hydrophilic and hydrophobic drugs. To date, there are several strategies to improve drug delivery efficiency through the TDDS, which may be broadly classified into four areas.

The first is a gel-type drug formulation. Such a formulation has the advantage of requiring no sophisticated system or platform. A gelating agent, which is mainly a polymer, and a drug form a three-dimensional network to reduce the mobility of molecules and encapsulate the drug. Since the gel formulation allows the drug to be absorbed into the skin using passive diffusion, the encapsulated drug needs to have a low molecular weight (less than about 600 Da) with sufficient hydrophobicity and be effective for low-dose administration.

Among various types of gelling agents, chitosan, a natural polymer containing a high ratio of glucosamine in its structure, has been widely used in gel-type drug formulations due to non-toxicity and sensitivity to degradation. The chitosan is positively charged under mildly acidic conditions and may reduce the cell membrane potential by depolarizing the negatively charged cell membrane, and as a result, has an advantage of being able to induce absorption of active ingredients or drugs, but has a disadvantage of causing degradation of the encapsulated drug in the presence of proteolytic enzymes dissolved at physiological pH due to partial protonation of the amino group of chitosan.

The second is nano/micro-sized liposomes or particles. These particles may be used for encapsulation of hydrophilic and hydrophobic drugs due to a unique structure consisting of a polar head and a nonpolar tail. The particles are absorbed into the skin by fusing the stratum corneum with the lipid bilayer, and the smaller the particle size, the higher the specific surface area and the more contact points with the skin, which is advantageous for the drug to penetrate the skin.

Hyaluronic acid, an anionic polysaccharide consisting of N-acetyl-glucosamine and glucuronic acid, has been widely used in liposome particles due to its moisturizing properties and biocompatibility. The hyaluronic acid-based nanoliposome has an advantage of high particle stability due to electrostatic, steric, and hydrophobic interactions with drugs and biomolecules and excellent permeability to stratum corneum due to the flexibility of a liposome structure but has a disadvantage that increased hydration of the skin surface may promote retention of the drug within the more hydrated epidermal layer, thereby limiting drug delivery to the dermal layer.

The third is a transdermal patch type, and the transdermal patch is a medicinal adhesive patch attached to the skin to deliver a specific dose of drug into the bloodstream through the skin or spread the drug to the skin, and includes an inert polymer that provides a support and platform for drug release, a drug dissolved or dispersed in a matrix, and a flexible support membrane that provides good bonding to a drug reservoir.

Poly(dimethylsiloxane) (PDMS) is the most widely used in transdermal patch formulations, including a reservoir, a matrix, and a drug-adhesive layer due to its inert and non-toxic properties. However, the PDMS has a disadvantage of limiting the release of the drug by strongly binding to the encapsulated drug due to its strong hydrophobicity.

Finally, a microneedle is a method of delivering drugs by penetrating the stratum corneum of the skin at a depth of 50 μm to 900 μm using a micron-sized needle and has an advantage of reducing patient pain and delivering the drug accurately to a target depth due to the small needle size. Polymers used to manufacture the microneedle need to have high water-solubility, biocompatibility, and mechanical strength for insertion into the skin.

Poly(lactide-co-glycolide) (PLGA) is widely used as a microneedle material due to its mechanical strength, high loading efficiency, biodegradability, and biocompatibility. The PLGA has an advantage that its degradation products are water-soluble and non-toxic, and the degradation rate of the polymer can be controlled depending on a chain length (molecular weight) and a lactide/glycolide ratio but has a disadvantage that PLGA-based microneedles need to remain inserted into the skin for several days for the inserted drug to be released.

As described above, the polymers used in conventional TDDS each have disadvantages, so that there is a need to develop a new type of polymer that can combine the advantages of the polymers and replace each disadvantage.

### DETAILED DESCRIPTION

### Technical Problem

One object of the present invention is to provide a silicone polymer compound represented by the following Chemical Formula 1:

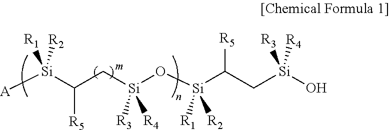

in which,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy,
R₅ is hydrogen or alkyl, and
A is

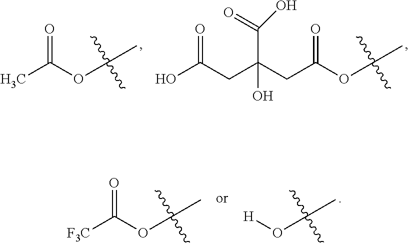

Another object of the present invention is to provide a method for preparing a silicone polymer compound represented by Chemical Formula 1 above, including ring-opening polymerizing a monomer represented by Chemical Formula A below using an initiator:

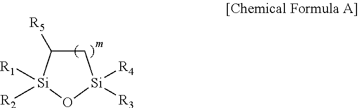

in which,
m is an integer of 1 to 3,
R₁ to R₄ are each independently any one selected from the group consisting of alkyl, vinyl, acetyl, aromatic, azide, hydroxy, and alkylsiloxy, and
R₅ is hydrogen or alkyl.

Another object of the present invention is to provide a composition for transdermal delivery of an active substance including a silicone polymer compound represented by Chemical Formula 1 above.

Another object of the present invention is to provide a method for transdermal delivery of an active substance, including topically applying the composition for transdermal delivery and the active substance.

Another object of the present invention is to provide an anti-cancer composition including a silicone polymer compound represented by Chemical Formula 1 above.

Another object of the present invention is to provide a method for preventing or treating cancer, including topically applying the anti-cancer composition.

Technical objects to be achieved in the present invention are not limited to the aforementioned objects, and other technical objects not described above will be apparently understood to those skilled in the art from the following disclosure of the present invention.

### Technical Solution

In order to achieve the object, an aspect of the present invention provides a silicone polymer compound represented by Chemical Formula 1 below:

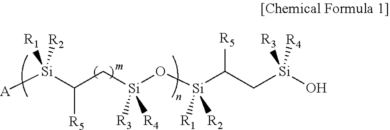

in which,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy,
R₅ is hydrogen or alkyl, and
A is

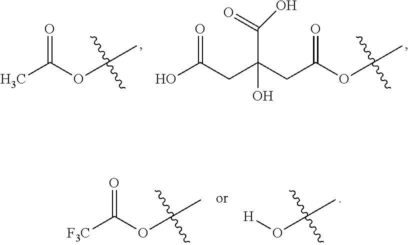

Another aspect of the present invention provides a method for preparing a silicone polymer compound represented by Chemical Formula 1 above, including ring-opening polymerizing a monomer represented by Chemical Formula A below using an initiator:

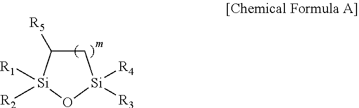

in which,
m is an integer of 1 to 3,
R₁ to R₄ are each independently any one selected from the group consisting of alkyl, vinyl, acetyl, aromatic, azide, hydroxy, and alkylsiloxy, and
R₅ is hydrogen or alkyl.

Another aspect of the present invention provides a composition for transdermal delivery of an active substance including a silicone polymer compound represented by Chemical Formula 1 above.

Another aspect of the present invention provides a method for transdermal delivery of an active substance, including topically applying the composition for transdermal delivery and the active substance.

Another aspect of the present invention provides an anti-cancer composition including a silicone polymer compound represented by Chemical Formula 1 above.

Another aspect of the present invention provides a method for preventing or treating cancer, including topically applying the anti-cancer composition.

### Advantageous Effects

According to the present invention, the silicone polymer compound has an advantage of being used as a delivery system loaded with drugs and various substances due to characteristics capable of controlling the molecular weight, viscosity, contact angle, moisture content, drug encapsulation ratio, and release behavior of a synthesized silicone polymer compound by controlling polymerization conditions such as the type of initiator, ratio of initiator to monomer, and polymerization temperature, etc. using the same monomer.

Further, the silicon polymer compound of the present invention has excellent biocompatibility without toxicity and can be used as a carrier for a new drug delivery system for transdermal absorption, by confirming high absorption and penetration rates compared to a control group, when confirming the efficiency of drug delivery through a transdermal absorption route by encapsulating a drug model in a polymer.

Furthermore, the silicon polymer compound of the present invention has excellent biocompatibility without showing toxicity or hemolysis even in a tumor model and can be used as an agent for cancer treatment by confirming high weight loss inhibition and tumor size/volume reduction effects compared to a control group and anticancer agent-alone application, when confirming a cancer treatment effect through a transdermal absorption route by encapsulating an anticancer agent.

It should be understood that the effects of the present invention are not limited to the effects described above but include all effects that can be deduced from the detailed description of the present invention or configurations of the invention described in claims.

### DESCRIPTION OF DRAWINGS

FIG. 1 is a schematic diagram showing a synthesis mechanism of a silicone polymer compound according to an embodiment of the present invention.

FIG. 2 is a graph of analyzing the structures of compounds A2, C2, T2, and H2 in a silicone polymer (PDDS) series and a monomer TDOP of an embodiment of the present invention using FI-TR.

FIG. 3 is a graph of analyzing the structures of a silicone polymer (PDDS) series (A, C, T, and H series) and a monomer of an embodiment of the present invention using FI-TR.

FIG. 4 is a graph of analyzing the structures of compounds C2, T2, and H2 in a silicone polymer (PDDS) series and a monomer TDOP of an embodiment of the present invention using ¹H NMR (CDCl₃).

FIG. 5 is a graph of analyzing the structures of a silicone polymer (PDDS) series (C, T, and H series) and a monomer of an embodiment of the present invention using ¹H NMR (CDCl₃).

FIG. 6 is a graph of analyzing the molecular weights of compounds C2, T2, and H2 in the silicone polymer (PDDS) series of an embodiment of the present invention using GPC.

FIG. 7 is a graph of analyzing the molecular weight of each of the silicone polymer (PDDS) series (C, T, and H series) of an embodiment of the present invention using GPC.

FIG. 8 is a graph of analyzing the viscosity of compounds C2, T2, and H2 in the silicone polymer (PDDS) series of an embodiment of the present invention using a Rheometer.

FIG. 9 is a graph of analyzing the viscosity of each of the silicone polymer (PDDS) series (C, T, and H series) of an embodiment of the present invention using a Rheometer.

FIG. 10 is an image of compounds C2, T2, and H2 in the silicone polymer (PDDS) series and a monomer of an embodiment of the present invention.

FIG. 11 is an image of compounds C1 to C3, T1 to T3, and H1 to H3 in the silicone polymer (PDDS) series and a monomer of an embodiment of the present invention.

FIG. 12 is an image of compounds C4 to C6, T4 to T6, and H4 to H6 in the silicone polymer (PDDS) series and a monomer of an embodiment of the present invention.

FIG. 13 is a graph of measuring contact angles of compounds C2, T2, and H2 in the silicone polymer (PDDS) series and a monomer of an embodiment of the present invention.

FIG. 14 is a graph of measuring contact angles a silicone polymer (PDDS) series (C, T and H series) and a monomer of an embodiment of the present invention.

FIG. 15 is a graph of measuring moisture contents of compounds C2, T2, and H2 in the silicone polymer (PDDS) series of an embodiment of the present invention.
(a) of FIG. 16 is a graph of a standard curve obtained by measuring the absorbance according to a concentration of oil red O which is a hydrophobic drug, and (b) of FIG. 16 is a graph of a standard curve obtained by measuring the absorbance according to a concentration of methylene blue which is a hydrophilic drug.

FIG. 17 is a graph of measuring an oil red O encapsulation ratio (%) and a graph of measuring a methylene blue encapsulation ratio (%) of compounds C2, T2, and H2 in the silicone polymer (PDDS) series of an embodiment of the present invention ((a) and (b), respectively).
(a) of FIG. 18 is an image of formulations encapsulated with oil red O in compounds C2, T2, and H2 in the silicone polymer (PDDS) series and a monomer of an embodiment of the present invention, (b) of FIG. 18 is an image of formulations encapsulated with methylene blue in compounds C2, T2, and H2 in the silicone polymer (PDDS) series and a monomer of an embodiment of the present invention, and (c) of FIG. 18 is an image when irradiating UV (365 nm) light to formulations encapsulated with Oil red O as a hydrophobic drug, doxorubicin as a hydrophilic anticancer agent, SN-38 as a hydrophobic anticancer agent, a peptide represented by a CGKRK sequence, and ciprofloxacin as an antibiotic into the compound C2 in the silicone polymer (PDDS) series of an embodiment of the present invention and C2 alone.

FIG. 19 is a graph of measuring a release curve of oil red O encapsulated in the silicone polymer (PDDS) series of an embodiment of the present invention in phosphate-buffered saline (PBS).

FIG. 20 is a graph of measuring a release curve of methylene blue encapsulated in the silicone polymer (PDDS) series of an embodiment of the present invention in phosphate-buffered saline (PBS).

FIG. 21 is a graph of measuring a change in body weight (a), a feed intake (b), and a water intake (c) of a mouse after treatment with PBS, C2 compound, and eugenol for 6 days in an embodiment of the present invention.

FIG. 22 is an image (a) photographing an auricular lymph node of a mouse and a graph (b) measuring a weight thereof after treatment with PBS, C2 compound, and eugenol for 6 days in an embodiment of the present invention.

FIG. 23 is a graph of measuring a BrdU concentration (a) and a stimulation index (b) of an auricular lymph node of a mouse through Bromodeoxyuridine assay after treatment with PBS, C2 compound, and eugenol for 6 days in an embodiment of the present invention.

FIG. 24 is a graph of measuring amounts of histamine (a) and immunoglobulin E (b) in blood of a mouse after treatment with PBS, C2 compound, and eugenol for 6 days in an embodiment of the present invention.

FIG. 25 is a graph of confirming the absorption rate of oil red O in skin tissue 24 hours (a to c) and 48 hours (e to f) after transdermal administration of an oil red O only substance and formulations in which oil red O was encapsulated into a C2 compound, in an embodiment of the present invention.

FIG. 26 is a graph of measuring a change in body weight (a) and a change in tumor volume (b) of the mouse after topically applying PBS and a C2 compound to a melanoma xenograft mouse and a graph (c) of confirming hemolysis by measuring the UV/vis absorption peak (λmax: 492 nm) according to treatment with PBS, C2 compound, and Triton-X to the mouse blood sample, in an embodiment of the present invention.

FIG. 27 is a schematic diagram (a) showing an experimental method of topically applying PBS, SN-38 and C2+SN-38 to a melanoma xenograft mouse, a graph of measuring a change in body weight (b) and a change in tumor volume (c) for 16 days thereafter, an image (d) of the mouse body and extracted tumor at the end of the experiment, a graph (e) showing the average volume of the extracted tumor, and a graph (f) of confirming hemolysis by measuring the UV/vis absorption peak (λmax: 492 nm) according to treatment with PBS, SN-38, C2+SN-38, and Triton-X to the mouse blood sample.

### BEST MODE OF THE INVENTION

Hereinafter, the present invention will be described in more detail. However, the present invention may be embodied in various different forms, and the present invention is not limited by embodiments described herein, and the present invention will be only defined by claims to be described below.

Terms used in the present invention are used only to describe specific embodiments and are not intended to limit the present invention. Throughout the present specification, unless explicitly described to the contrary, ‘comprising’ a certain component means further comprising another component other than excluding the other component. In addition, the ‘comprising’ above also means using a component alone.

One aspect of the present invention provides a silicone polymer compound and a method for preparing the same.

The present inventors developed a poly(dimethylsilylethylene-dimethylsiloxane) (PDDS) series which is a silicone polymer compound with a structure similar to polydimethylsiloxane (PDMS) through ring-opening polymerization of monomers with a cyclic siloxane structure, in order to synthesize a new type of polymer capable of replacing the disadvantages of polymers used in a conventional transdermal delivery system (TDDS) and combining the advantages.

The PDDS series of the present invention has a structure similar to PDMS, and is characterized by having the high biocompatibility, hydrophobicity, and high viscosity characteristics of PDMS, and including both hydrophilic and hydrophobic properties by introducing a large amount of hydroxyl groups (—OH) contained in chitosan and hyaluronic acid at the terminal.

It was confirmed that the chemical and mechanical properties (molecular weight, viscosity, contact angle, etc.) of the PDDS series developed in the present invention were variously controlled depending on a type of acidic initiator, a ratio of monomers to initiators, a polymerization temperature, etc., and due to these properties, the drug encapsulation efficiency and drug release behavior varied for each PDDS series. In addition, even when the silicone polymer of an embodiment of the present invention not only showed low toxicity and excellent biocompatibility in mice, but also confirmed skin absorption efficiency, it was confirmed that compared to the control group, the drug was absorbed into the skin for a longer period of time and passed through the dermis layer of the skin to deliver the drug to the subcutaneous tissue, and then the present invention was completed.

The silicone polymer compound provided by the present invention, that is, the PDDS series described above, may be represented by Chemical Formula 1 below:

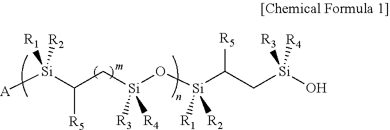

in which,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy,
R₅ is hydrogen or alkyl, and
A is

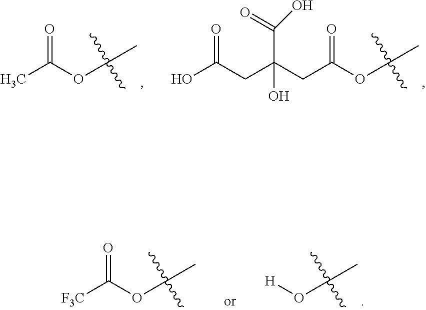

An aspect of the present invention also provides a method for preparing a silicone polymer compound including ring-opening polymerizing a monomer represented by Chemical Formula A below using an initiator:

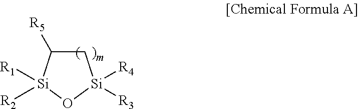

in which,
m is an integer of 1 to 3,
R₁ to R₄ are each independently any one selected from the group consisting of alkyl, vinyl, acetyl, aromatic, azide, hydroxy, and alkylsiloxy, and
R₅ is hydrogen or alkyl.

The silicone polymer compound of the present invention may be prepared using the method for preparing the silicone polymer compound.

In an embodiment of the present invention, the initiator may include organic acids, organic sulfonic acids, inorganic acids, etc., and specifically, may be at least one selected from the group consisting of trifluoroacetic acid, phosphoric acid, fumaric acid, acetic acid, alkyl sulfonic acid having 1 to 12 carbon atoms, sulfonic acid, hydrochloric acid, phosphoric acid, carboxylic acid, sulfonic acid, and citric acid, for example, any one of acetic acid, trifluoroacetic acid, citric acid or hydrochloric acid, for example, any one of acetic acid, citric acid or hydrochloric acid, and preferably either citric acid or hydrochloric acid.

Depending on the type of the initiator, A indicated in Chemical Formula 1 above may be different.

For example, when the initiator is citric acid, A in Chemical Formula 1 above may be

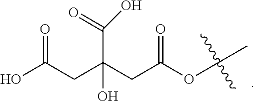 [SMILES: *OC(=O)CC(O)(CC(=O)O)C(=O)O]

As another example, when the initiator is trifluoroacetic acid, A in Chemical Formula 1 above may be

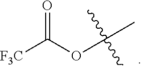 [SMILES: *OC(=O)C(F)(F)F]

As yet another example, when the initiator is hydrochloric acid, A in Chemical Formula 1 above may be

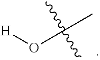 [SMILES: *O]

As another example, when the initiator is acetic acid, A in Chemical Formula 1 above may be

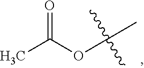 [SMILES: *OC(C)=O]

In a specific embodiment of the present invention, the monomer represented by Chemical Formula A may be 2,2,5,5-tetramethyl-2,5-disila-1-oxacyclopentane represented by Chemical Formula A′ below:

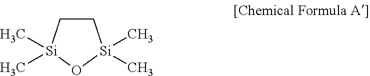

When the monomer of the silicone polymer compound of the present invention is 2,2,5,5-tetramethyl-2,5-disila-1-oxacyclopentane represented by Chemical Formula A′ above, in Chemical Formula 1 above, m may be 1, R₁ to R₄ may be all methyl groups, and R₅ may be hydrogen.

In a specific embodiment of the present invention, when the monomer represented by Chemical Formula A is 2,2,5,5-tetramethyl-2,5-disila-1-oxacyclopentane and the initiator is citric acid, the silicone polymer compound of the present invention may include a silicone polymer represented by Chemical Formula 2 below:

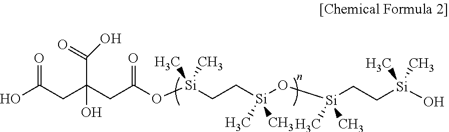

in which,
n is an integer of 10 to 35.

In a specific embodiment of the present invention, when the monomer represented by Chemical Formula A is 2,2,5,5-tetramethyl-2,5-disila-1-oxacyclopentane and the initiator is trifluoroacetic acid, the silicone polymer compound of the present invention may include a silicone polymer represented by Chemical Formula 3 below:

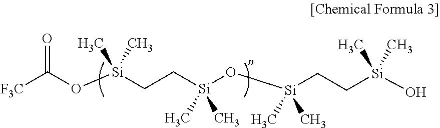

in which,
n is an integer of 55 to 335.

In a specific embodiment of the present invention, when the monomer represented by Chemical Formula A is 2,2,5,5-tetramethyl-2,5-disila-1-oxacyclopentane and the initiator is hydrochloric acid, the silicone polymer compound of the present invention may include a silicone polymer represented by Chemical Formula 4 below:

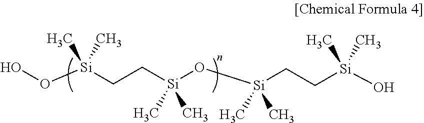

in which,
n is an integer of 55 to 335.

In a specific embodiment of the present invention, when the monomer represented by Chemical Formula A is 2,2,5,5-tetramethyl-2,5-disila-1-oxacyclopentane and the initiator is acetic acid, the silicone polymer compound of the present invention may include a silicone polymer represented by Chemical Formula 5 below:

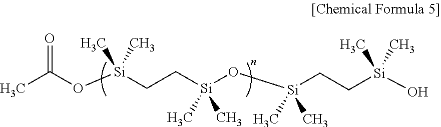

in which,
n is an integer of 1 to 10.

In an embodiment of the present invention, in the ring-opening polymerization step, the physical properties of the prepared silicone polymer compound may be controlled by controlling at least any one of a type of initiator, a molar ratio of initiator to monomer, and a polymerization temperature.

At this time, the physical properties of the prepared silicone polymer compound may be at least any one of molecular weight, viscosity, contact angle, and moisture content.

In an embodiment of the present invention, the type of initiator that may be used in the preparation method of the present invention is as described above, and the molecular weight, viscosity, and/or moisture content of the silicone polymer compound prepared according to the type of initiator may be controlled.

Specifically, as the acidity of the initiator is stronger, both the molecular weight and viscosity of the prepared silicone polymer compound may increase, and as the acidity of the initiator is weaker, the moisture content may increase.

In an embodiment of the present invention, the initiator may be added at a molar ratio of 0.01 to 1.5 based on the monomer, but the addition ratio of the initiator may vary depending on the type of the above-described initiator.

For example, when the initiator is citric acid, the initiator may be included in a molar ratio of 0.01 to 1.0, for example, 0.01 to 0.5, for example, 0.01 to 0.3, for example, 0.01 to 0.2, based on the monomer.

As another example, when the initiator is trifluoroacetic acid, the initiator may be included in a molar ratio of 0.01 to 1.5, for example, 0.01 to 1.4, for example, 0.02 to 1.3, based on the monomer.

As yet another example, when the initiator is hydrochloric acid, the initiator may be included in a molar ratio of 0.01 to 1.0, for example, 0.01 to 0.8, for example, 0.02 to 0.7, based on the monomer.

As yet another example, when the initiator is acetic acid, the initiator may be included in a molar ratio of 0.01 to 1.0, for example, 0.01 to 0.5, for example, 0.01 to 0.3, for example, 0.01 to 0.2, based on the monomer.

In an embodiment of the present invention, the molecular weight and/or viscosity of the prepared silicone polymer compound may be controlled depending on the molar ratio of initiator to monomer.

Specifically, as the ratio of the initiator to the monomer decreases, both the molecular weight and viscosity of the prepared silicone polymer compound may increase.

In an embodiment of the present invention, the polymerization conditions such as time and temperature of the polymerization step may be set according to the physical properties of the silicone polymer compound to be synthesized, and the polymerization step may be performed, for example, at a temperature of 10° C. to 100° C., for example, 10° C. to 90° C., for example, 10° C. to 80° C., for example, 20° C. to 80° C., for example, 25° C. to 80° C. for 10 hours to 100 hours, for example, 10 hours to 80 hours, for example, 10 hours to 60 hours, and 10 hours to 50 hours, for example, 20 hours to 50 hours.

In an embodiment of the present invention, the molecular weight and/or viscosity of the prepared silicone polymer compound may be controlled depending on the polymerization temperature conditions.

Specifically, as the polymerization temperature increases, both the molecular weight and viscosity of the prepared silicone polymer compound may increase.

In an embodiment of the present invention, the contact angle of the prepared silicone polymer compound may be controlled according to a type of initiator described above, a ratio of initiator to monomer, and/or a polymerization temperature condition.

Specifically, when the prepared silicone polymer compound is the compound represented by Chemical Formula 2 above, as the ratio of initiator to monomer decreases and the polymerization temperature increases, the contact angle may increase, and when the prepared silicone polymer compound is the compound represented by Chemical Formula 3 or 4 above, as the ratio of initiator to monomer decreases, the contact angle may increase.

As described above, the physical properties of the prepared silicone polymer compound may be controlled by controlling at least any one of a type of initiator, a molar ratio of initiator to monomer, and a polymerization temperature.

In a specific embodiment, the silicone polymer compound of the present invention may be loaded with drugs having various characteristics, such as hydrophobic model drugs, hydrophilic model drugs, hydrophilic anticancer agents, hydrophobic anticancer agents, peptides, antibiotics, etc. At this time, the characteristics of an active substance (drug) to be encapsulated, an encapsulation ratio of the drug, and/or release behavior of the encapsulated drug may also be controlled according to at least any one of the type of initiator, the molar ratio of initiator to monomer, and the polymerization temperature and vary according to physical properties of the prepared silicone polymer compound.

Therefore, it is possible to design and prepare the silicone polymer compound of the present invention according to the use, method, and drug characteristics.

In an embodiment of the present invention, the molecular weight of the silicone polymer compound prepared using the preparation method may be 1,000 g/mol to 100,000 g/mol, but the molecular weight of the silicone polymer compound may vary according to the type of initiator described above.

For example, when the initiator is citric acid, the molecular weight of the silicone polymer compound may be 1,000 g/mol to 100,000 g/mol, for example, 1,000 g/mol to 10,000 g/mol, for example, 1,000 g/mol to 8,000 g/mol, for example, 1,000 g/mol to 6,000 g/mol, but is not limited thereto.

As another example, when the initiator is trifluoroacetic acid, the molecular weight of the silicone polymer compound may be 1,000 g/mol to 100,000 g/mol, for example, 5,000 g/mol to 100,000 g/mol, for example, 5,000 g/mol to 80,000 g/mol, for example, 9,000 g/mol to 8,000 g/mol, but is not limited thereto.

As yet another example, when the initiator is hydrochloric acid, the molecular weight of the silicone polymer compound may be 1,000 g/mol to 100,000 g/mol, for example, 5,000 g/mol to 100,000 g/mol, for example, 10,000 g/mol to 100,000 g/mol, for example, 30,000 g/mol to 9,000 g/mol, but is not limited thereto.

In an embodiment of the present invention, the viscosity of the silicone polymer compound prepared using the preparation method may be 60 cP to 120,000 cP, but the viscosity of the silicone polymer compound may vary according to the type of initiator described above.

For example, when the initiator is citric acid, the viscosity of the silicone polymer compound may be 60 cP to 120,000 cP, for example, 60 cP to 10,000 cP, for example, 60 cP to 1,000 cP, for example, 60 cP to 600 cP, but is not limited thereto.

As another example, when the initiator is trifluoroacetic acid, the viscosity of the silicone polymer compound may be 60 cP to 120,000 cP, for example, 100 cP to 100,000 cP, for example, 500 cP to 100,000 cP, for example, 600 cP to 96,000 cP, but is not limited thereto.

As yet another example, when the initiator is hydrochloric acid, the viscosity of the silicone polymer compound may be 60 cP to 120,000 cP, for example, 100 cP to 120,000 cP, for example, 1,000 cP to 118,000 cP, for example, 1,000 cP to 115,000 cP, but is not limited thereto.

In an embodiment of the present invention, the water contact angle of the silicone polymer compound prepared using the preparation method may be 490 or higher, but the water contact angle of the silicone polymer compound may vary according to the type of initiator described above.

For example, when the initiator is citric acid, the water contact angle of the silicone polymer compound may be 490 or higher, for example, 490 to 100°, for example, 49° to 90°, for example, 49° to 85°, but is not limited thereto.

As another example, when the initiator is trifluoroacetic acid, the water contact angle of the silicone polymer compound may be 490 or higher, for example, 600 to 110°, for example, 800 to 105°, for example, 850 to 102°, but is not limited thereto.

As yet another example, when the initiator is hydrochloric acid, the water contact angle of the silicone polymer compound may be 490 or higher, for example, 600 to 115°, for example, 800 to 110°, for example, 900 to 107°, but is not limited thereto.

Another aspect of the present invention provides a composition for transdermal delivery of an active substance including a silicone polymer compound represented by Chemical Formula 1 below:

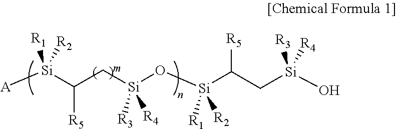

in which,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy, and
R₅ is hydrogen or alkyl, and
A is

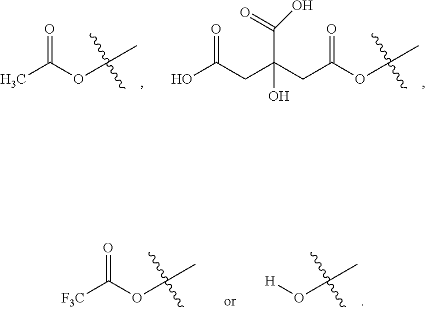

Another aspect of the present invention provides a method for transdermal delivery of an active substance, including topically applying the composition for transdermal delivery and the active substance.

In an embodiment of the present invention, A may be

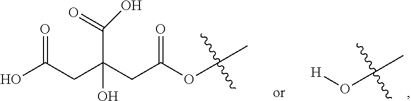

and n may be an integer of 10 to 35 or 240 to 505.

In an embodiment of the present invention, the active substance may be a transdermal agent or a transdermal preparation to be transdermally administered, and the composition for transdermal delivery may increase transdermal adsorption of the active substance.

In an embodiment of the present invention, the composition for transdermal delivery may further include an active substance requiring transdermal delivery, for example, a preparation that requires increased transdermal absorption rate and transdermally administered, and the transdermal absorption may be absorption in the epidermis, dermis, and hypodermis.

The active substance may be loaded into the silicone polymer compound of the present invention to increase the transdermal absorption rate, and specifically, the active substance may be loaded into a chain structure of the polymer compound to increase the transdermal absorption rate.

The term ‘loading’ may be used interchangeably with terms such as ‘supporting’ and ‘encapsulating’, and may mean that the polymer compound and the active substance are mixed to be physically or chemically bound, but is not limited thereto.

In an embodiment of the present invention, the silicone polymer compound may have a high loading rate, that is, a high encapsulation ratio, of the active substance, and specifically, the active substance may be loaded at an encapsulation ratio of 10% to 99%, for example, 10% to 90%.

In an embodiment of the present invention, when the silicone polymer compound is loaded with the active substance and administered transdermally, for example, the active substance may be applied to the skin and continue to be absorbed for 3 hours or more, 6 hours or more, 12 hours or more, 24 hours or more, or 48 hours or more after skin application. At this time, even if the upper limit of the absorption retention time of the active substance is not specified, those skilled in the art will clearly understand the characteristics of the polymer compound of the present invention that improves the skin absorption retention of the active substance, but the absorption retention time may be, for example, 240 hours or less, 200 hours or less, 168 hours or less, 150 hours or less, 120 hours or less, 100 hours or less, or 72 hours or less, but is not limited thereto.

In an embodiment of the present invention, the active substance may be included without limitation as long as the active substance is a preparation administered transdermally that requires improvement or increase in transdermal absorption rate, and may be, for example, a pharmaceutical composition, a cosmetic composition, a diagnostic reagent composition, or an imaging agent.

In addition, the active substance may be at least any one of a hydrophobic active substance, a hydrophilic active substance, a peptide, and an antibiotic, and the type of active substance loaded into the silicone polymer compound may also be designed and determined according to the physical properties of the silicone polymer compound.

In an embodiment of the present invention, the pharmaceutical composition may be at least one formulation selected from the group consisting of ointment, cream, gel, lotion, spray, patch, spray, emulsion, and suspension, but is not limited thereto, and the cosmetic composition may be at least one formulation selected from the group consisting of skin, lotion, cream, essence, solution, external ointment, foam, lotion, nutritional lotion, softening lotion, pack, toner, emulsion, sunscreen cream, sun oil, suspension, emulsion, paste, gel, powder, serum, emulsion, essence, foundation, and mask pack, but is not limited thereto.

In an embodiment of the present invention, the active substance may be mixed with the silicone polymer of the present invention to form a physical or chemical bond, thereby improving the transdermal absorption rate.

Another aspect of the present invention provides an anti-cancer composition including a silicone polymer compound represented by Chemical Formula 1 below:

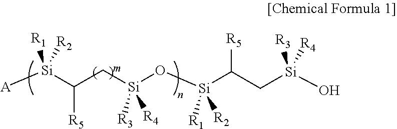

in which,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy,
R₅ is hydrogen or alkyl, and
A is

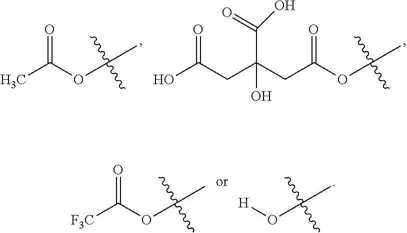

Another aspect of the present invention provides a method for preventing or treating cancer, including topically applying the anti-cancer composition.

In an embodiment of the present invention, A may be

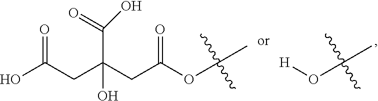

and n may be an integer of 10 to 35 or 240 to 505.

In an embodiment of the present invention, the anti-cancer composition may further include an anticancer agent, and further include at least any one anticancer agent selected from the group consisting of, for example, 7-ethyl-10-hydroxycamptothecin (SN-38), irinotecan, doxorubicin, paclitaxel, epirubicin, gemcitabine, cilolimus, etoposide, vinblastine, vinca alkaloid, docetaxel, cisplatin, Gleevec, adriamycin, cyclophosphamide, teniposide, 5-fluorouracil, camptothecin, tamoxifen, anasterozole, floxuridine, leuprolide, flotamide, zoledronate, vincristine, streptozotocin, carboplatin, ifosfamide, topotecan, belotecan, irinotecan, vinorelbine, hydroxyurea, valrubicin, methotrexate, mechlorethamine, chlorambucil, busulfan, doxifluridine, prednisone, testosterone, mitoxantrone, docetaxel, vinorelbine, and prednisolone, and preferably irinotecan or its active metabolite, SN-38.

In an embodiment of the present invention, in the anti-cancer composition, an anticancer agent may be loaded into the silicone polymer compound. The anticancer agent may be loaded into the silicone polymer compound of the present invention to increase the transdermal absorption rate, and specifically, the anticancer agent may be loaded into a chain structure of the polymer compound to increase the transdermal absorption rate. The transdermal absorption may be absorption in the epidermis, dermis, and hypodermis. The loading may mean that the polymer compound and the anticancer agent are mixed to be physically or chemically bound, but is not limited thereto.

In an embodiment of the present invention, the silicone polymer compound may have a high loading rate, that is, a high encapsulation ratio, of the anticancer agent, and specifically, the anticancer agent may be loaded at an encapsulation ratio of 10% to 99%, for example, 10% to 90%.

In an embodiment of the present invention, in the anti-cancer composition, the anticancer agent may be loaded into the silicone polymer compound to exhibit the effect of increasing the anticancer effect of an anticancer agent even without showing the toxicity and hemolytic of the anticancer agent. Specifically, the cancer treatment effect of reducing the size and volume of cancer or tumor may be increased. The cancer treatment effect may include the effect of suppressing weight loss caused by cancer.

In an embodiment of the present invention, the silicone polymer compound included in the anti-cancer composition may be used as an anti-cancer adjuvant. The anticancer adjuvant may be used to increase the anticancer treatment effect of the anticancer agent and to suppress or improve the side effects of the anticancer agent when administered to the patient in combination with the anticancer agent.

In an embodiment of the present invention, the anti-cancer composition may be, for example, a transdermal agent or a transdermal preparation which is transdermally administered, and may be formulated without limitation as long as it is a preparation administered transdermally for cancer treatment, and may be a pharmaceutical composition, an external skin composition, or a cosmetic composition.

In an embodiment of the present invention, the cancer may be any one selected from the group consisting of skin cancer, thyroid cancer, stomach cancer, colon cancer, lung cancer, liver cancer, breast cancer, prostate cancer, gallbladder cancer, biliary tract cancer, pancreatic cancer, oral cancer, esophageal cancer, bladder cancer, colon cancer, and cervical cancer. The skin cancer may include melanoma, squamous cell carcinoma, basal cell carcinoma, etc.

In an embodiment of the present invention, the method for the prevention or treatment of cancer includes administering the anti-cancer composition in a therapeutically effective amount to a subject. It is preferred that a specific therapeutically effective amount for a specific subject is differently applied depending on various factors including the kind and degree of a response to be achieved, a specific composition including whether other agents are used in some cases, the age, body weight, general health conditions, sex, and diet of a subject, an administration time, an administration route, a secretion rate of the composition, a duration of treatment, and a drug used in combination or simultaneously with the specific composition, and similar factors well known in the medical field. Therefore, the effective amount of the composition suitable for the purpose of the present invention is preferably determined in consideration of the aforementioned matters.

In an embodiment of the present invention, the method for the prevention or treatment of cancer may be administering the anti-cancer composition to a subject in need of treatment, the subject in need of the treatment may mean a patient, the subject is applicable to any mammal, and the mammal includes not only humans and primates, but also livestock such as cattle, pigs, sheep, horses, dogs and cats.

### Modes of the Invention

Hereinafter, the present disclosure will be described in more detail through Examples. However, the following Examples are just illustrative of the present invention, and the contents of the present invention are not limited to the following Examples.

### EXAMPLES

### Example 1. Synthesis of Silicone Polymer (PDDS) Series

Through a ring-opening polymerization reaction of 2,2,5,5-tetramethyl-2,5-disila-1-oxacyclopentane as a monomer and acetic acid (AA), citric acid (CA), trifluoroacetic acid (TFA) or hydrochloric acid (HCl) as an initiator, a silicon-based polymer (poly(dimethylsilylethylene-dimethylsiloxane); hereinafter referred to as PDDS) series was synthesized.

Synthesis was performed by varying a type of initiator, a ratio of monomer to initiator, and/or a polymerization temperature condition of the PDDS series, and the reaction conditions were summarized and shown in Table 1 below:

TABLE 1

| | PDDS | | [monomer]:[initiator] | Temperature |
|---|---|---|---|---|
| Series | name | Initiator | (molar ratio) | (° C.) |
| A series | A1 | AA | 61.3:1 | 80 |
| | A2 | AA | 12.3:1 | 80 |
| | A3 | AA | 6.1:1 | 80 |
| | A4 | AA | 61.3:1 | 25 |
| | A5 | AA | 12.3:1 | 25 |
| | A6 | AA | 6.1:1 | 25 |
| C series | C1 | CA | 63.3:1 | 80 |
| | C2 | CA | 13.8:1 | 80 |
| | C3 | CA | 6.9:1 | 80 |
| | C4 | CA | 63.3:1 | 25 |
| | C5 | CA | 13.8:1 | 25 |
| | C6 | CA | 6.9:1 | 25 |
| T series | T1 | TFA | 40.8:1 | 80 |
| | T2 | TFA | 4.1:1 | 80 |
| | T3 | TFA | 0.8:1 | 80 |
| | T4 | TFA | 40.8:1 | 25 |
| | T5 | TFA | 4.1:1 | 25 |
| | T6 | TFA | 0.8:1 | 25 |
| H series | H1 | HCl | 44.1:1 | 80 |
| | H2 | HCl | 4.8:1 | 80 |
| | H3 | HCl | 1.5:1 | 80 |
| | H4 | HCl | 44.1:1 | 25 |
| | H5 | HCl | 4.8:1 | 25 |
| | H6 | HCl | 1.5:1 | 25 |

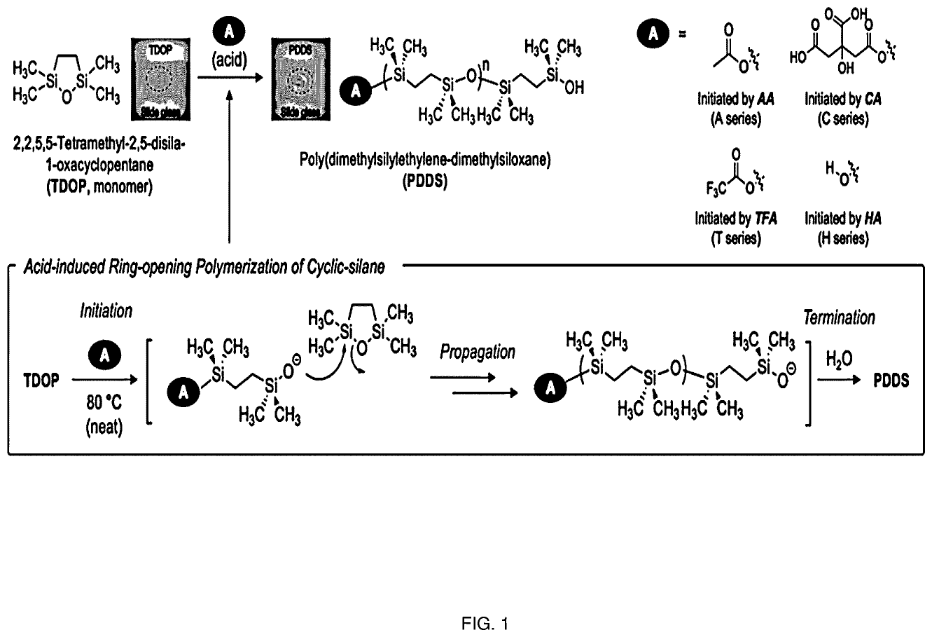

FIG. 1 shows a schematic diagram of a synthesis mechanism of a C2 compound in a silicone polymer series of the present invention. Referring to Table 1 and FIG. 1, when specifically describing the synthesis of the C2 compound, 2,2,5,5-tetramethyl-2,5-disila-1-oxacyclopentane (200 μL, 1.07 mmol) was added to a 4 mL glass vial containing 2 M citric acid (40 μL, solvent: ethanol), reacted at 80° C. for 48 hours while stirring at a speed of 1,000 rpm, added with a small amount of water to terminate the reaction, washed three times with ethanol (1 mL) to remove unreacted 2,2,5,5-tetramethyl-2,5-disila-1-oxacyclopentane and citric acid and purified, and dissolved in n-hexane (200 μL) and then dried in an oven at 80° C. overnight to obtain the C2 compound.

In the method for synthesizing the C2 compound, the type of initiator, the ratio of monomer to initiator, and/or the polymerization temperature condition were varied according to the conditions disclosed in Table 1, compounds A1 to A6, C1, C3 to C6, T1 to T6 and H1 to H6 were synthesized and obtained, respectively.

### Experimental Example

### Experimental Example 1. Characteristic Analysis

### (1) Chemical Structure Analysis

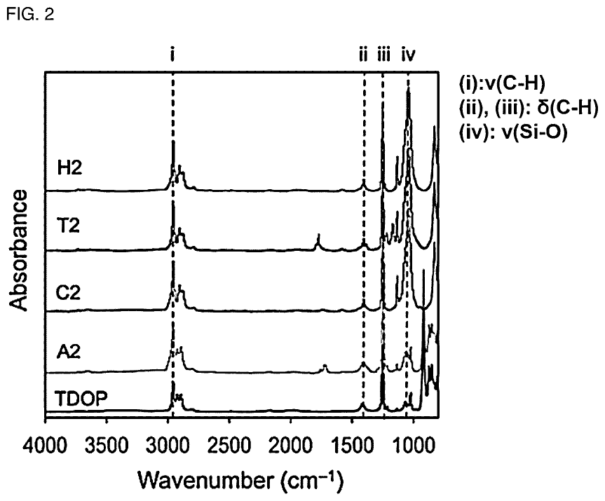

The chemical structures of the PDDS series compounds synthesized in Example 1 were analyzed using Fourier-transform infrared spectroscopy (FT-IR) and nuclear magnetic resonance (NMR), and the analysis results were shown in FIGS. 2 to 5.

Meanwhile, in the case of A series compounds prepared using acetic acid (AA) as an initiator, polymers were not formed well, so that FT-IR and NMR data were not clearly obtained, and thus, in the following Experimental Examples, specific characteristics and effects were confirmed for C, T, and H series compounds excluding the A series.

### FT-IR Analysis

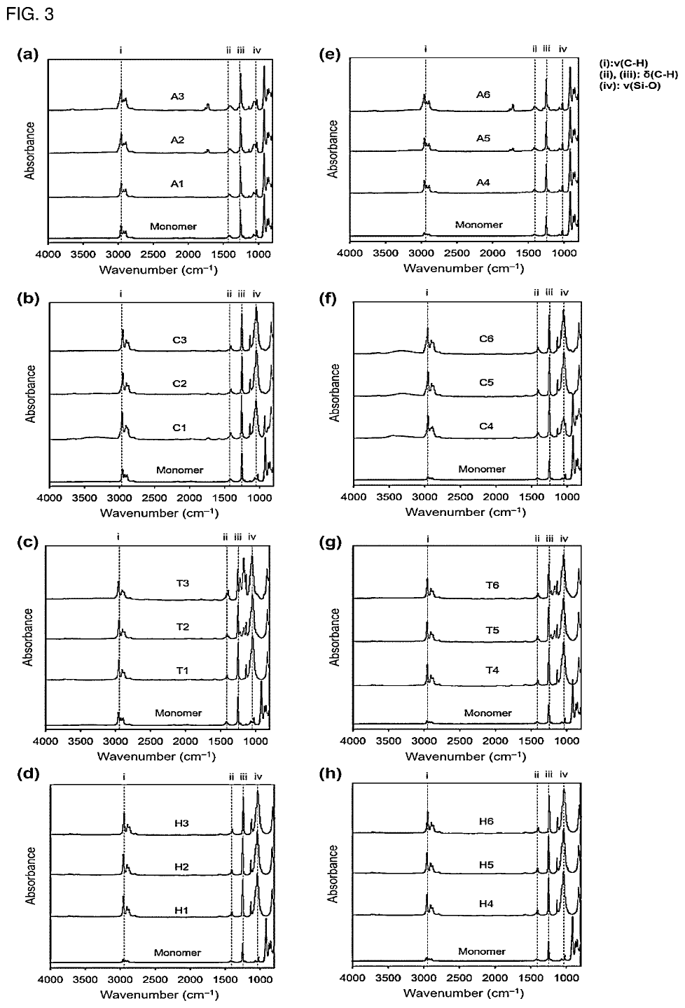

FIGS. 2 and 3 show the results of analyzing PDDS series functional group according to an embodiment of the present invention using Fourier-transform infrared spectroscopy and the analysis was performed using attenuated total reflection Fourier transform infrared (FT-IR) spectroscopy of Thermo Fisher Co., Ltd.

Referring to FIGS. 2 and 3, in the case of monomers, ν(C—H) peaks of alkane at 2,950 cm⁻¹ to 2,850 cm⁻¹, δ(C—H) peaks of methyl group and dimethyl group at 1,450 cm⁻¹ and 1,385 cm⁻¹ to 1,380 cm⁻¹, and ν(Si—O) peaks at 1,065 cm⁻¹ were identified (ν=stretching, 6=bending), respectively, and a silane functional group (Si—O—Si) and a silicon carbide (Si—C) functional group contained in a monomer structure were identified. Even in the case of the PDDS series, it was confirmed that only the intensity of ν increased at 1,065 cm¹, and there was the same functional group as the monomer, and thus the polymer was formed without a change in the monomer structure during polymerization.

### NMR Analysis

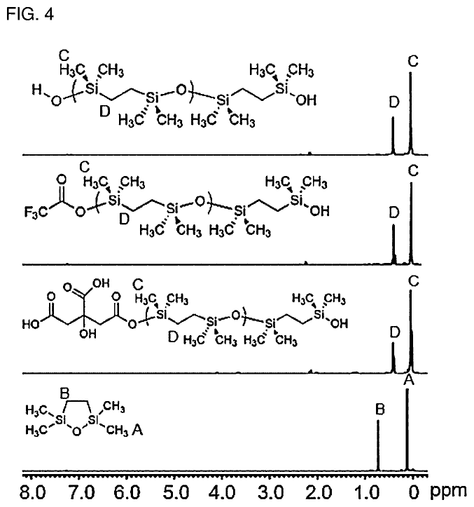

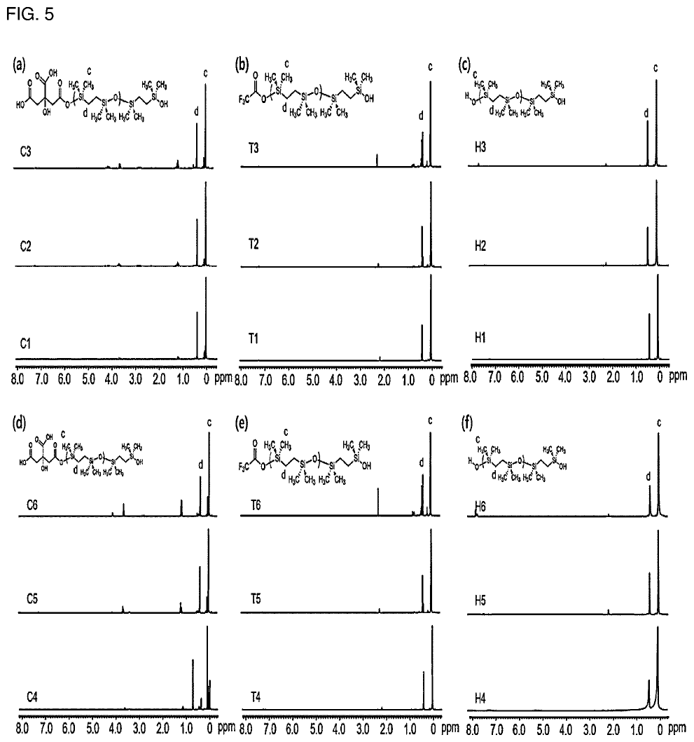

FIGS. 4 and 5 show the results of nuclear magnetic resonance (NMR) analysis of the PDDS series. Specifically, an analysis sample was prepared by dissolving the monomer and the polymer in a CDCl₃ (67.26 ppm) solution, and ¹H NMR was measured at a temperature of 25° C. using an NMR spectrometer FT-NMR 500 MHz (JEOL product). The number of measurements was 16, and the peak of CDCl₃ was set at 7.26 ppm to set a reference point.

Referring to FIGS. 4 and 5, as a result of NMR analysis of the monomer, a hydrogen atom (proton, 1H) in Si—CH₃ at 0.313 ppm and a hydrogen atom in Si—CH₂ at 0.745 ppm were found, and as a result of analyzing the PDDS series, it was confirmed that the positions of hydrogen atoms moved to 0.020 ppm and 0.414 ppm, respectively. It could be expected that the reason was that while the ring structure of the monomer was opened, the interactions between molecules were changed.

### (2) Molecular Weight and Viscosity Analysis

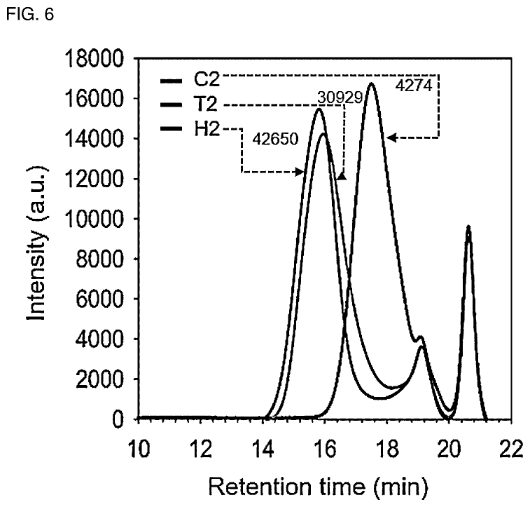

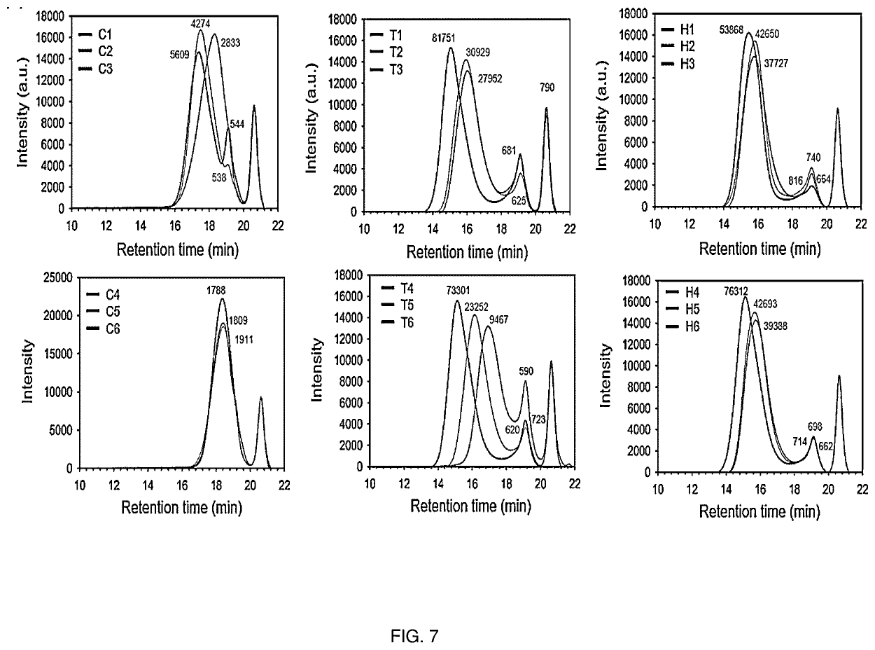

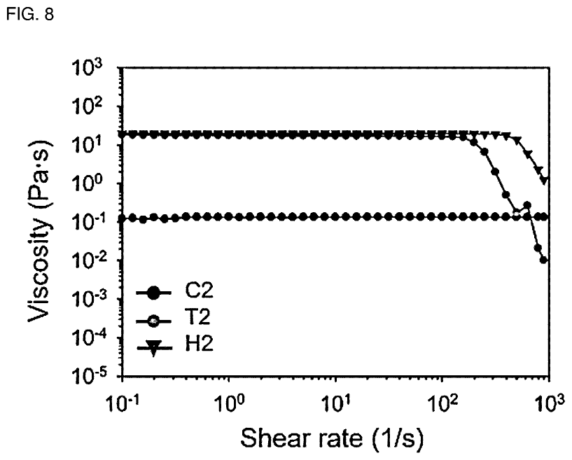

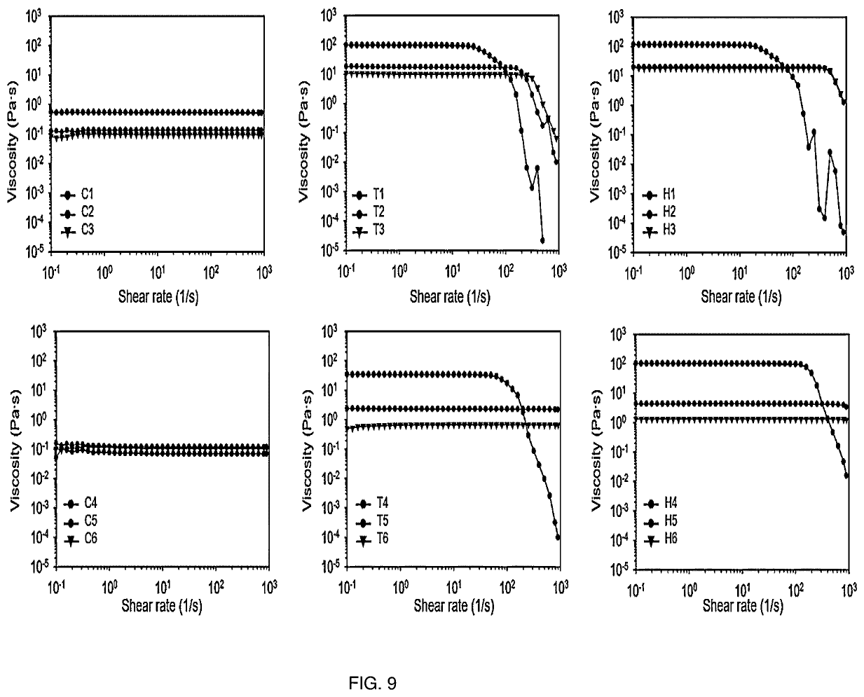

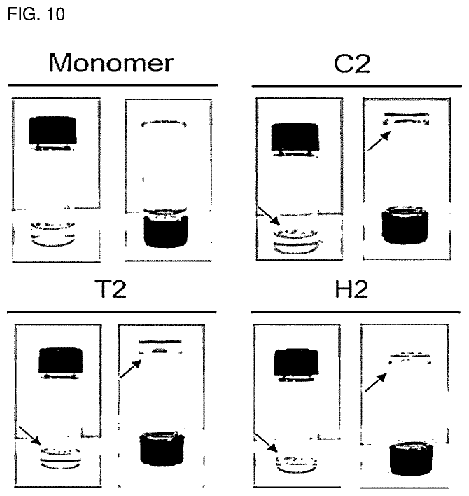

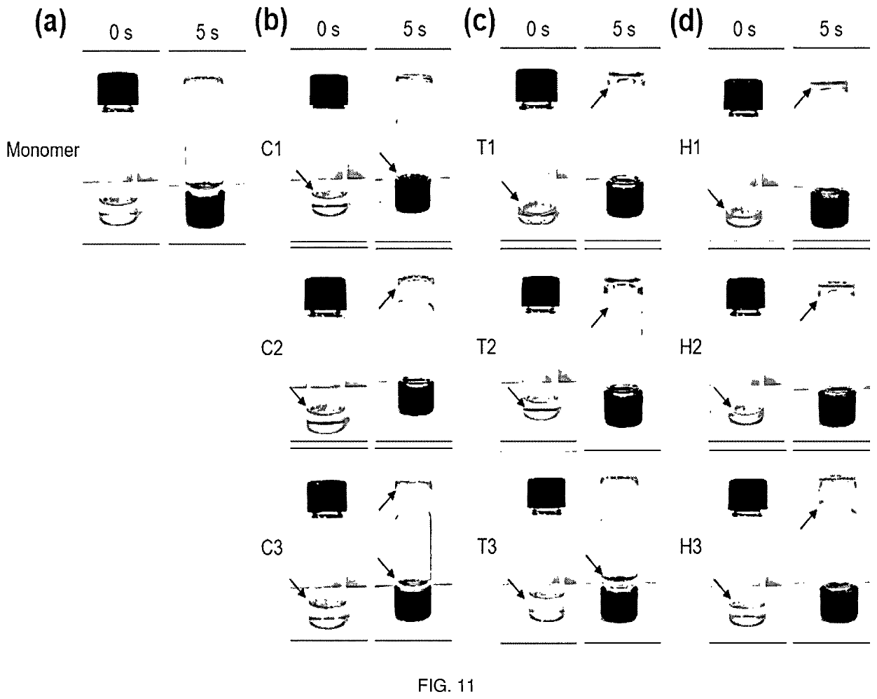

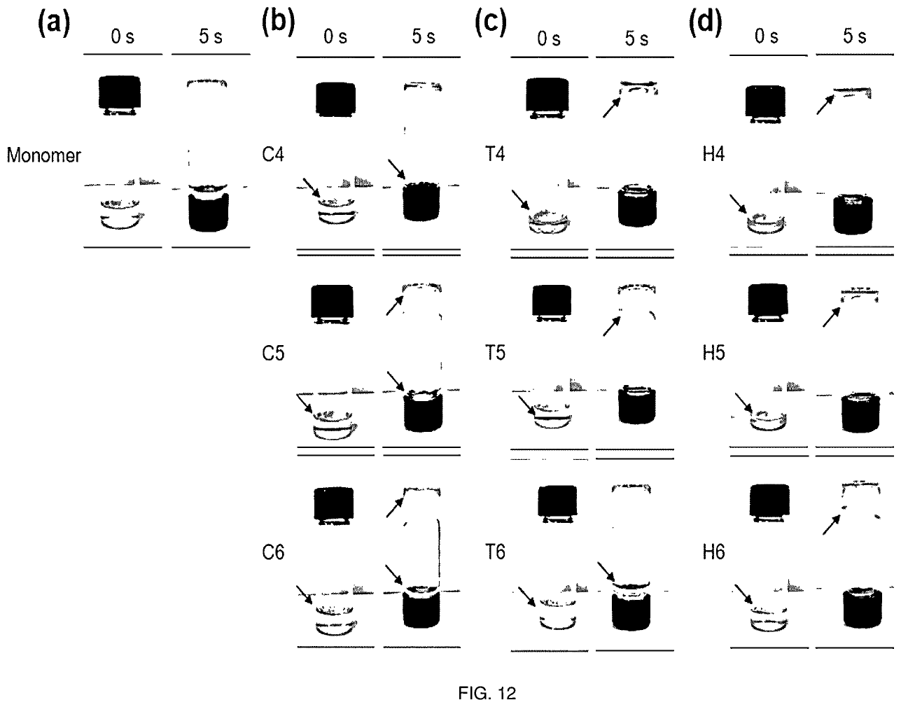

The molecular weight and viscosity of the PDDS series synthesized in Example 1 were measured using gel permeation chromatography (GPC) and a rotational rheometer, and then the respective measurement results were shown in FIGS. 6 to 9. The monomer and C2, T2, and H2 of the PDDS series were added in a glass vial, and then images in the forward and reverse directions were measured and shown in FIGS. 10 to 12.

Referring to FIGS. 6 to 12, from the GPC results, it was confirmed that the molecular weight of PDDS increased depending on the acidity of the initiator. Specifically, it was confirmed that C2 had a molecular weight of 4,274 g/mol (Polydisperisty index; PDI: 2.07) and a conversion rate of 86.6%, T2 had a molecular weight of 30,929 g/mol (PDI: 1.75) and a conversion rate of 96.9%, and H2 had a molecular weight of 42,650 g/mol (PDI: 1.60) and a conversion rate of 96.6%.

It was confirmed that the molecular weight of the PDDS series increased as the ratio of monomer to initiator increased and slightly decreased at a high reaction temperature (80° C.), but under all polymerization conditions, the PDDS series showed a narrow molecular weight distribution range of 1.40<Mw/Mn<2.07 and a high conversion rate (>85%). In addition, it was confirmed that all the PDDS series showed increased viscosity compared to the monomer and showed higher viscosity at a strongly acidic initiator, a high ratio of monomer to initiator, and a low polymerization temperature, similarly to the molecular weight results.

### (3) Contact Angle and Moisture Content Analysis

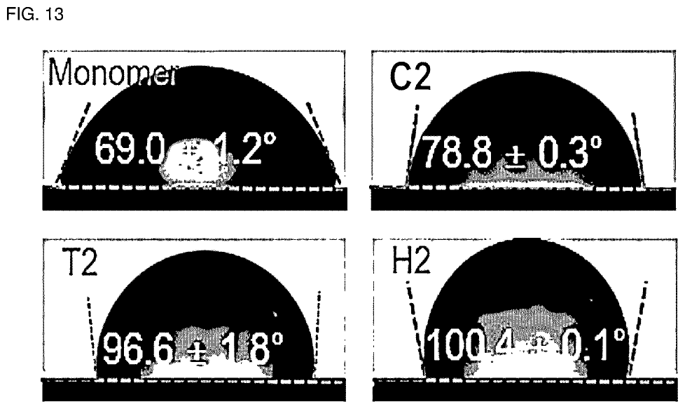

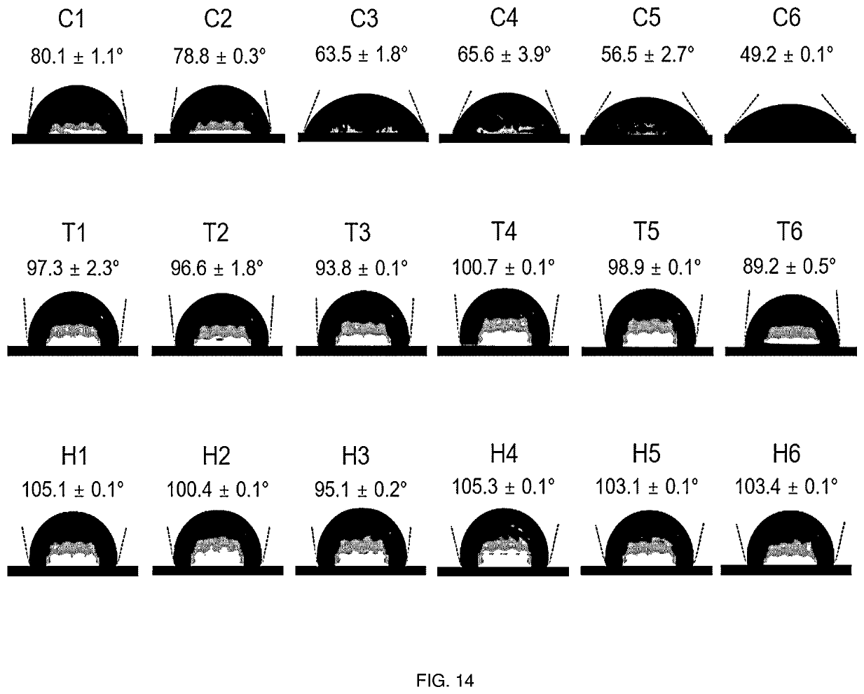

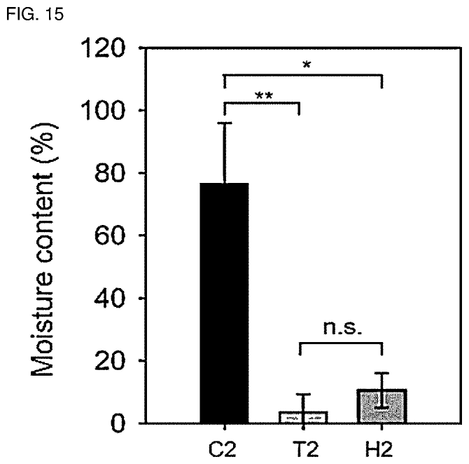

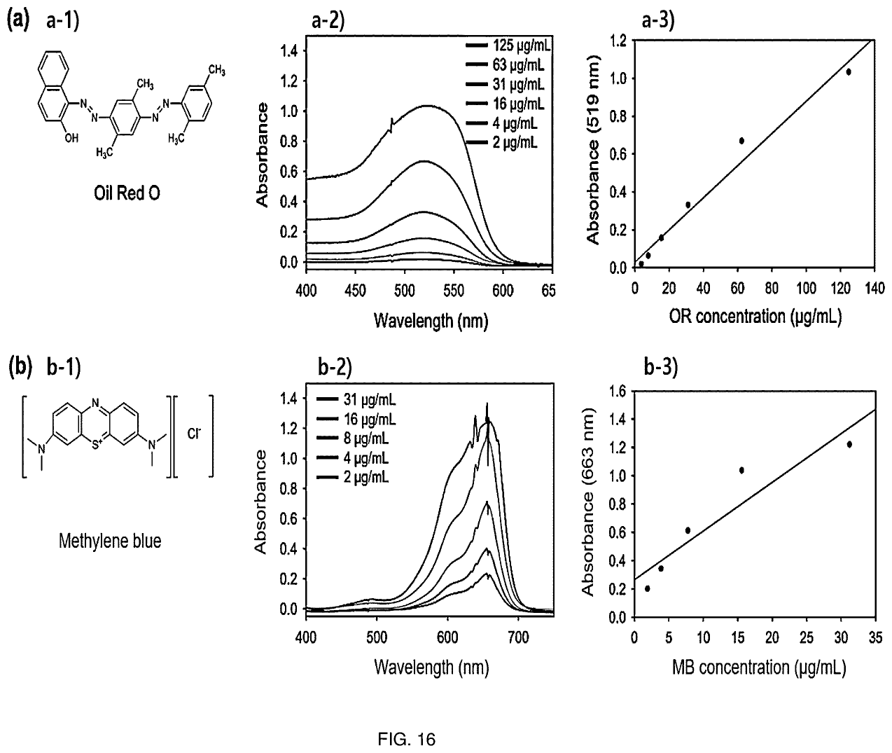

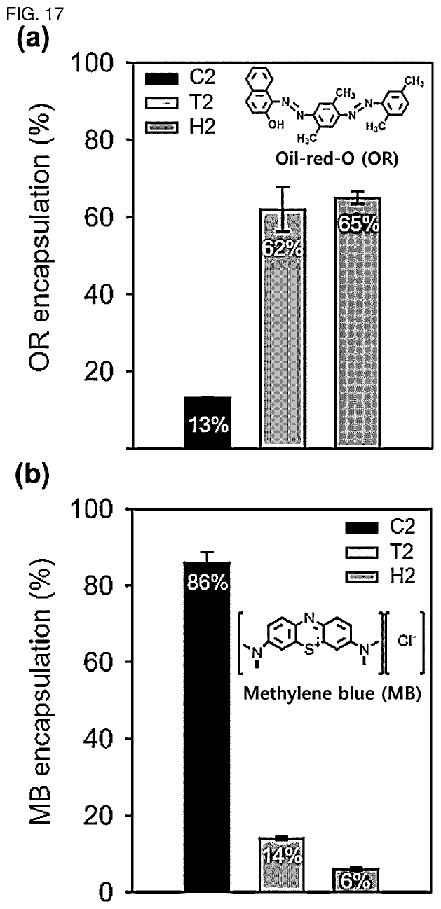

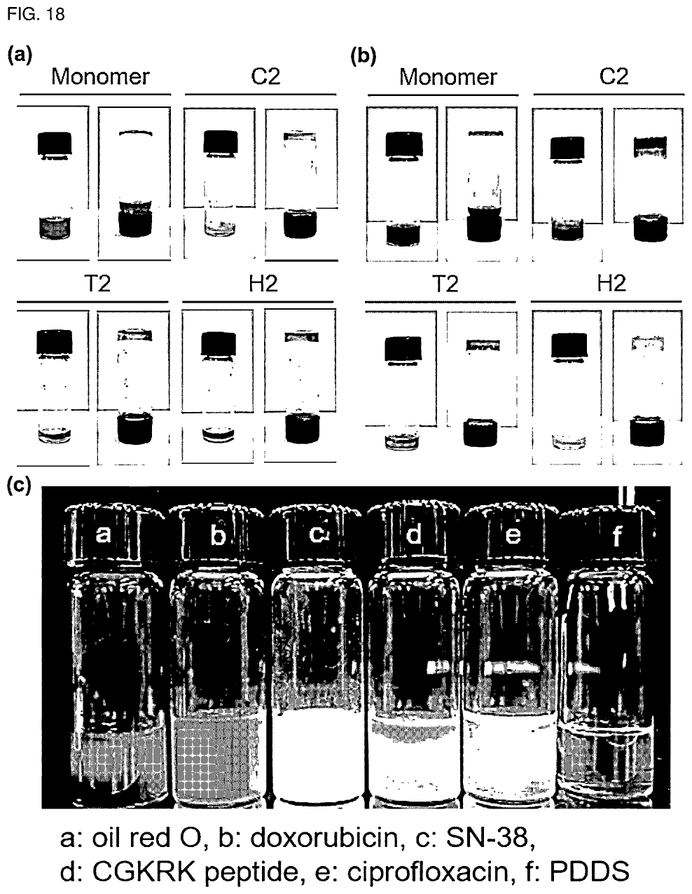

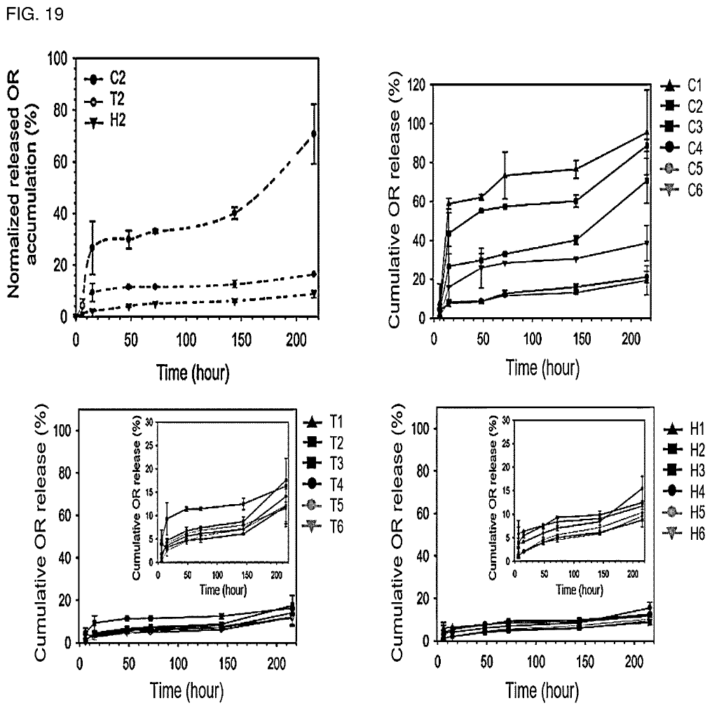

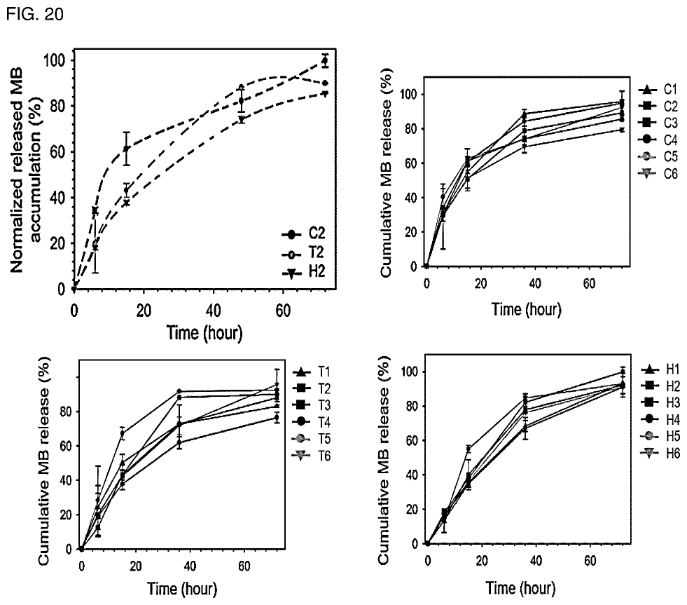

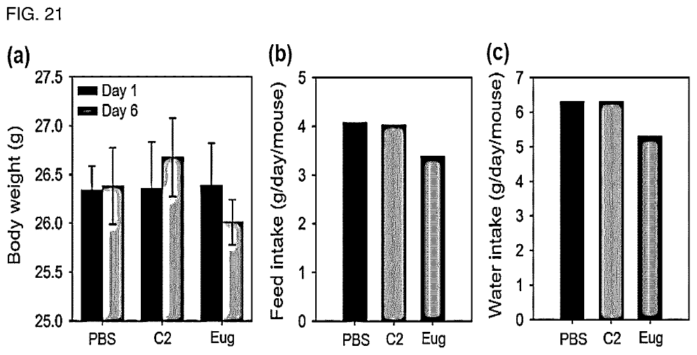

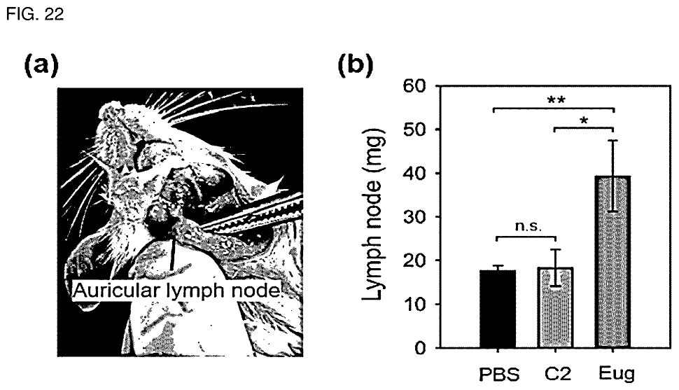

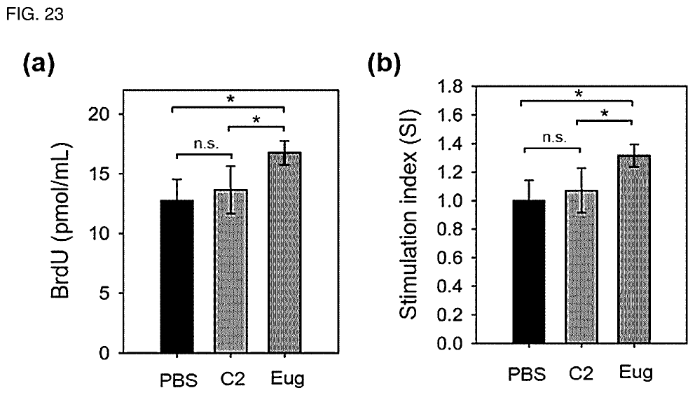

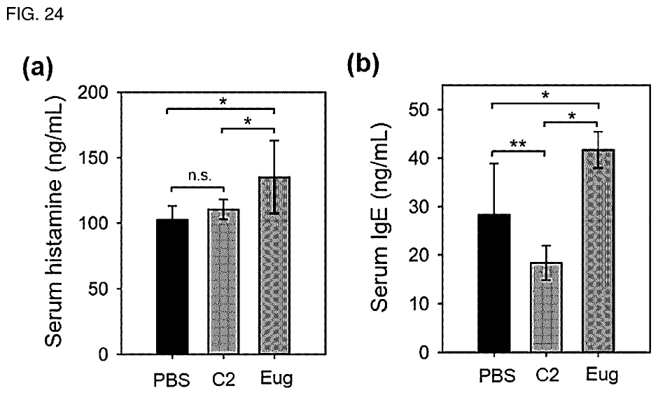

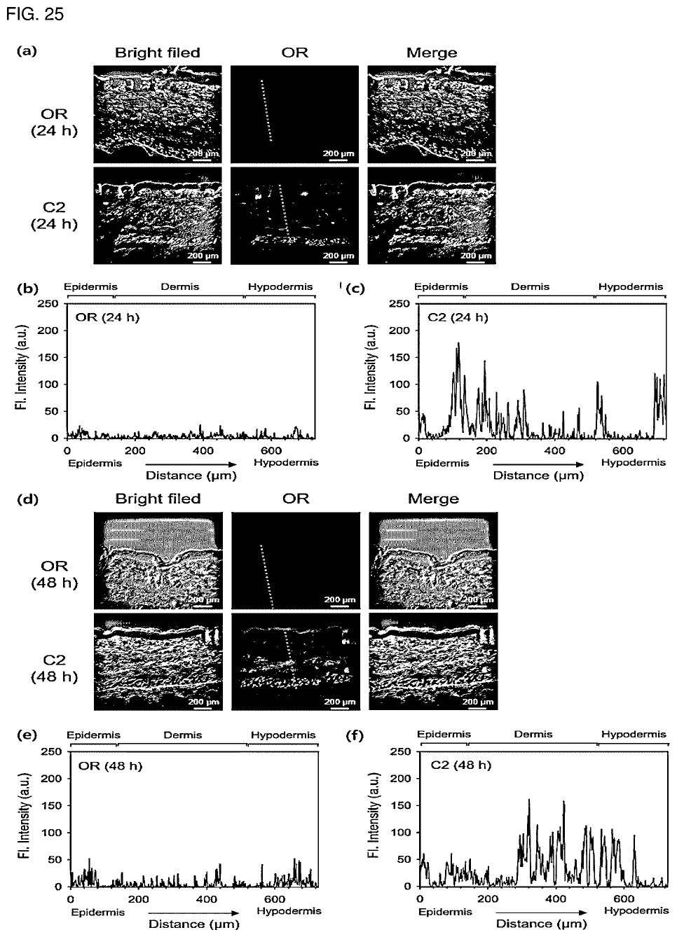

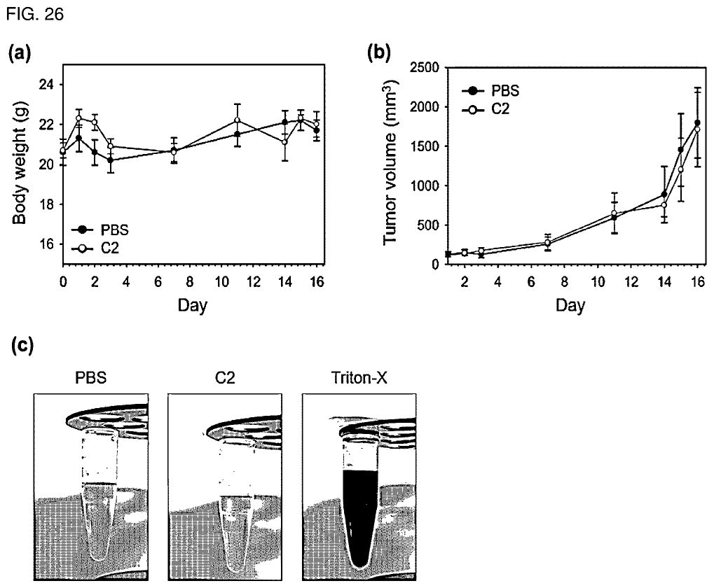

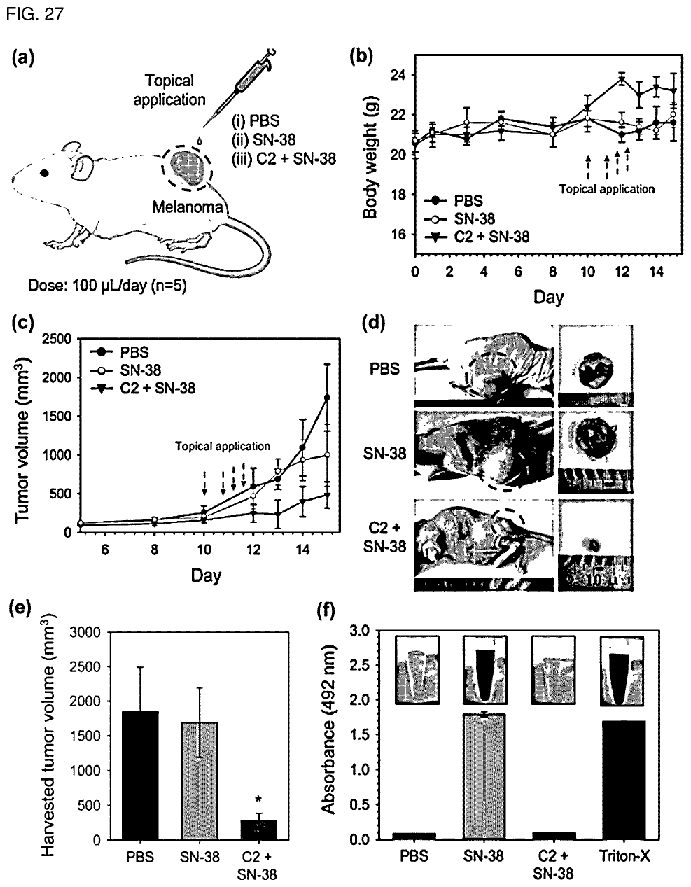

FIGS. 13 and 14 show results of analyzing the contact angles of the monomer and the PDDS series of Example 1.

Specifically, a monomer and PDDS series compounds were applied onto a slide glass from Marienfeld using a 100 μL pipette, respectively, and then the monomer and the PDDS series were uniformly coated on the slide glass while the slide glass was tilted left and right. The monomer and the PDDS series were coated for about 1 hour in a fume hood, and then 100 μL each of the monomer and PDDS series were dropped again, and coated without an empty portion of the slide glass by repeating the process two more times. Thereafter, distilled water was dropped on the surface of the coated glass slide using a contact angle meter from Kruss and the contact angles were analyzed.

Referring to FIGS. 13 and 14, as a result of measuring the contact angles, it was confirmed that the contact angle of the monomer was 69.0±1.2°, but the contact angle increased after polymerization progressed.

It was confirmed that the contact angle tended to increase as the acidity of the initiator became stronger, the ratio of initiator to monomer decreased, and the polymerization temperature increased, but specifically, polymers C4 to C6 synthesized at 25° C. using citric acid were more hydrophilic than the monomer. (C4: 65.6±3.9°, C5: 56.5±2.7°, C6: 49.2±0.10)

FIG. 15 is a graph of measuring moisture contents according to hydrophilicity/hydrophobicity through the contact angles of FIGS. 13 and 14 of the PDDS series. Specifically, 100 μL of each PDDS series was added in a 1.5 mL tube from Eppendorf, dried in a freeze dryer for 24 hours, and then weighed. Thereafter, 100 μL of water was added to each tube and mixed for 2 hours using a vortex mixer from Scientific Industries, and then left at room temperature for about 1 hour to separate water and PDDS layers. The remaining water layer was removed, and then the weights were measured again to measure the moisture contents.

Referring to FIG. 15, it was confirmed that the moisture content was similar to the contact angle results, and in the case of the highly hydrophobic PDDS series (T series and H series), a small moisture content of less than 10% was shown, but in the case of the C series, the moisture content was 76% of higher depending on the polymerization conditions.

The results of analyzing the characteristics of each compound in the PDDS series of Experimental Example 1 were summarized in Tables 2 and 3 below:

TABLE 2

| | | Mw | Mn | PDI | Conversion | Contact | Moisture |
|---|---|---|---|---|---|---|---|
| Series | PDDS | (g/mol) | (g/mol) | (Mw/Mn) | (%) | angle (°) | content (%) |
| C series | C1 | 5,609 | 4,011 | 1.40 | 97.4 | 80.1 ± 1.1 | 57.8 ± 13.2 |
| | C2 | 4,274 | 2,069 | 2.07 | 86.6 | 78.8 ± 0.3 | 76.5 ± 19.4 |
| | C3 | 2,833 | 1,412 | 2.01 | 85.0 | 63.5 ± 1.8 | 61.8 ± 25.5 |
| | C4 | 1,788 | 1,212 | 1.48 | 87.6 | 65.6 ± 3.9 | 45.2 ± 11.1 |
| | C5 | 1,911 | 1,205 | 1.59 | 87.7 | 56.5 ± 2.7 | 51.7 ± 15.5 |
| | C6 | 1,809 | 1,152 | 1.57 | 88.2 | 49.2 ± 0.1 | 54.5 ± 27.6 |
| T series | T1 | 53,868 | 33,793 | 1.59 | 100 | 97.3 ± 2.3 | 8.4 ± 16.6 |
| | T2 | 30,929 | 17,662 | 1.75 | 96.9 | 96.6 ± 1.8 | 3.5 ± 5.8 |
| | T3 | 27,952 | 16,972 | 1.65 | 99.7 | 93.8 ± 0.1 | 6.2 ± 3.6 |
| | T4 | 73,301 | 40,339 | 1.82 | 100 | 100.7 ± 0.1 | 7.2 ± 5.3 |
| | T5 | 23,252 | 14,178 | 1.64 | 100 | 98.9 ± 0.1 | 5.7 ± 2.1 |
| | T6 | 9,467 | 6,601 | 1.43 | 98.9 | 89.2 ± 0.5 | 10.6 ± 7.3 |
| H series | H1 | 81,751 | 44,658 | 1.83 | 99.7 | 105.1 ± 0.1 | 7.2 ± 4.2 |
| | H2 | 42,650 | 26,686 | 1.60 | 96.6 | 100.4 ± 0.1 | 10.5 ± 5.5 |
| | H3 | 37,727 | 21,815 | 1.73 | 99.6 | 95.1 ± 0.2 | 12.8 ± 9.7 |
| | H4 | 76,312 | 39,965 | 1.91 | 97.4 | 105.3 ± 0.1 | 8.0 ± 2.1 |
| | H5 | 42,693 | 25,286 | 1.69 | 99.9 | 103.1 ± 0.1 | 9.6 ± 4.3 |
| | H6 | 39,388 | 22,715 | 1.73 | 100 | 103.4 ± 0.1 | 7.9 ± 5.8 |

TABLE 3

| | Shear | Viscosity | Viscosity | | Shear | Viscosity | Viscosity |
|---|---|---|---|---|---|---|---|
| PDDS | rate (1/s) | (Pa · s) | (cP) | PDDS | rate (1/s) | (Pa · s) | (cP) |
| C1 | 1 | 0.534 | 534 | C4 | 1 | 0.076 | 76 |
| | 10 | 0.53 | 530 | | 10 | 0.07 | 70 |
| | 100 | 0.526 | 526 | | 100 | 0.069 | 69 |
| C2 | 1 | 0.136 | 136 | C5 | 1 | 0.116 | 116 |
| | 10 | 0.134 | 134 | | 10 | 0.105 | 105 |
| | 100 | 0.135 | 135 | | 100 | 0.105 | 105 |
| C3 | 1 | 0.122 | 122 | C6 | 1 | 0.096 | 96 |
| | 10 | 0.119 | 119 | | 10 | 0.095 | 95 |
| | 100 | 0.118 | 118 | | 100 | 0.095 | 95 |
| T1 | 1 | 34.0 | 34,000 | T4 | 1 | 95.6 | 95,600 |
| | 10 | 33.9 | 33,900 | | 10 | 93.5 | 93,500 |
| | 100 | 17.3 | 17,300 | | 100 | 11.8 | 11,800 |
| T2 | 1 | 17.4 | 17,400 | T5 | 1 | 2.32 | 2,320 |
| | 10 | 17.1 | 17,100 | | 10 | 2.3 | 2,300 |
| | 100 | 16.7 | 16,700 | | 100 | 2.29 | 2,290 |
| T3 | 1 | 10.2 | 10,200 | T6 | 1 | 0.655 | 655 |
| | 10 | 10 | 10,000 | | 10 | 0.676 | 676 |
| | 100 | 9.86 | 9,860 | | 100 | 0.676 | 676 |
| H1 | 1 | 114 | 114,000 | H4 | 1 | 101.5 | 101,500 |
| | 10 | 111 | 111,000 | | 10 | 101 | 101,000 |
| | 100 | 9.10 | 9,100 | | 100 | 97 | 97,000 |
| H2 | 1 | 19.7 | 19,700 | H5 | 1 | 4.35 | 4,350 |
| | 10 | 19.7 | 19,700 | | 10 | 4.35 | 4,350 |
| | 100 | 19.6 | 19,600 | | 100 | 4.34 | 4,340 |
| H3 | 1 | 18.5 | 18,500 | H6 | 1 | 1.33 | 1,330 |
| | 10 | 18.3 | 18,300 | | 10 | 1.33 | 1,330 |
| | 100 | 18.2 | 18,200 | | 100 | 1.32 | 1,320 |

### Experimental Example 2. Analysis of Drug Encapsulation Ratio and Drug Release Behavior

### Drug Encapsulation Ratio

In order to confirm various drug encapsulation ratios of the PDDS series synthesized in Example 1, oil red O as a hydrophobic drug model and methylene blue as a hydrophilic drug model were encapsulated, and the encapsulation efficiency and release behavior in phosphate-buffered saline (PBS) were analyzed, which were shown in related drawings, FIGS. 16 to 20.

To load the drug model, 100 μL of the PDDS series was added in a 4 mL vial, and then added with 1 mL of an oil red O stock (1 mg/mL ethanol) or a methylene blue stock (1 mg/mL ethanol). Using a stirrer, the polymer and the drug were mixed by stirring at room temperature (25° C.) for 24 hours to encapsulate the drug. After 24 hours, in order to remove the drug that was not encapsulated, 1 mL of ethanol was added, stirred for 10 minutes, and then the process of removing the supernatant was repeated three times.

In order to measure the encapsulation efficiency of the drug, the supernatant was collected from each washing process, and the encapsulation efficiency was analyzed through comparison with a standard curve by measuring an absorption spectrum of the drug model using a UV/Vis spectrophotometer from Agilent.

To obtain the standard curve, a solution of oil red O at a concentration of 2 μg/mL to 125 μg/mL dissolved in ethanol and a solution of methylene blue at a concentration of 2 μg/mL to 31 μg/mL dissolved in ethanol were prepared, and then the absorbance for the concentration was analyzed and shown in FIG. 16.

Specifically, (a) of FIG. 16 is a graph of a standard curve obtained by measuring the absorbance according to a concentration of oil red O which is a hydrophobic drug, and (b) of FIG. 16 is a graph of a standard curve obtained by measuring the absorbance according to a concentration of methylene blue which is a hydrophilic drug.

Referring to the standard curve of each drug model in FIG. 16, the maximum peak values of the PDDS series compounds of Example 1 were measured to calculate the standard curve of each drug.

The oil red O and methylene blue encapsulation ratios of the PDDS series of Example 1 were shown in Table 4 below, and a graph of measuring the encapsulation ratio (%) of oil red O of C2, T2 and H2 compounds in the PDDS series and a graph of measuring the encapsulation ratio (%) of methylene blue thereof were shown in FIG. 17 ((a) of FIG. 17 and (b) of FIG. 17, respectively).

TABLE 4

| | oil red O | methylene blue |
|---|---|---|
| PDDS | encapsulation ratio (%) | encapsulation ratio (%) |
| C1 | 10 ± 0.37 | 68 ± 2.77 |
| C2 | 13 ± 0.47 | 86 ± 2.77 |
| C3 | 12 ± 0.11 | 46 ± 2.72 |
| C4 | 27 ± 0.39 | 85 ± 8.93 |
| C5 | 34 ± 1.42 | 87 ± 6.60 |
| C6 | 38 ± 3.09 | 38 ± 3.09 |
| T1 | 62 ± 5.19 | 6 ± 0.64 |
| T2 | 62 ± 5.74 | 14 ± 0.46 |
| T3 | 61 ± 3.17 | 16 ± 1.09 |
| T4 | 79 ± 7.10 | 8 ± 1.54 |
| T5 | 57 ± 3.09 | 5 ± 0.22 |
| T6 | 58 ± 1.75 | 16 ± 1.26 |
| H1 | 70 ± 2.14 | 6 ± 0.21 |
| H2 | 65 ± 1.62 | 6 ± 0.36 |
| H3 | 63 ± 5.62 | 18 ± 1.47 |
| H4 | 70 ± 1.23 | 7 ± 0.48 |
| H5 | 63 ± 1.27 | 9 ± 0.54 |
| H6 | 63 ± 4.45 | 9 ± 0.24 |

Referring to Table 4 and FIG. 17, it was confirmed that the PDDS series of the present invention had different encapsulation ratios of each drug model depending on the type of initiator, the ratio of initiator to monomer, and the polymerization temperature. It was confirmed that according to the polymerization condition, in the case of oil red 0, the encapsulating efficiency of up to 79% or more was shown compared to the amount added (T4 in the PDDS series) and in the case of methylene blue, the encapsulating efficiency of up to 87% or more was shown compared to the amount added (C5 in the PDDS series) to control the encapsulation efficiency while successfully inducing the encapsulation of the drug. (a) of FIG. 18 is an image of formulations encapsulated with oil red O in a monomer and compounds C2, T2, and H2 in the silicone polymer (PDDS) series, (b) of FIG. 18 is an image of formulations encapsulated with methylene blue in a monomer and compounds C2, T2, and H2 in the silicone polymer (PDDS) series, and (c) of FIG. 18 is an image when irradiating UV (365 nm) light to formulations encapsulated with oil red O as a hydrophobic drug, doxorubicin as a hydrophilic anticancer agent, SN-38 as a hydrophobic anticancer agent, a peptide represented by a CGKRK sequence, and ciprofloxacin as an antibiotic into the compound C2 in the PDDS series and C2 alone.

Referring to (a) of FIG. 18 and (b) of FIG. 18, a noticeable decrease in viscosity was not confirmed in the PDDS series after the drug was loaded. Referring to (c) of FIG. 18, it was confirmed that even when the silicon polymer compound C2 of the present invention was encapsulated with a hydrophobic drug model (oil red O), a hydrophilic anticancer agent (doxorubicin), a hydrophobic anticancer agent (SN-38), a peptide (CGKRK peptide), and an antibiotic (ciprofloxacin), the encapsulation was successful.

FIGS. 19 and 20 are graphs of measuring release curves of oil red O (FIG. 19) and methylene blue (FIG. 20) encapsulated in the PDDS series in phosphate-buffed saline (PBS), respectively.

Specifically, 100 μL of the PDDS series encapsulated with oil red O and methylene blue were dispersed in 1 mL of PBS and incubated at 37° C. After a certain period of time, the PDDS series and the phase-separated PBS layer were obtained, measured with an absorption spectrum, and compared with the standard curve to quantify the amounts of oil red O and methylene blue released.

Referring to FIGS. 19 and 20, it was confirmed that the release behavior of the encapsulated drug may be controlled according to the PDDS series, and it could be seen that in the case of the T series and H series loaded with oil red 0, only about 10% of the encapsulated drug was released even after 9 days, whereas in the case of the C series, 70% was released. It could be seen that in the case of methylene blue, in most series, most of the drug was released after 3 days.

### Experimental Example 3. Confirmation of Biocompatibility (Confirmation of In Vivo Toxicity)

To confirm the biocompatibility of the PDDS series, immunotoxicity was confirmed using mice.

Specifically, 25 μL each of phosphate-buffered saline (PBS) as a negative control, eugenol (Eug) inducing immunotoxicity as a positive control, and a C2 compound in the PDDS series were applied behind the ears of mice for 3 days, and after 6 days, the change in body weight, the feed intake and water intake, the weight of auricular lymph nodes, the number of lymph node cells, the stimulation index, and the concentrations of histamine and immunoglobulin E in the blood were measured.

FIG. 21 is a graph of measuring a change in body weight (a), a feed intake (b), and a water intake (c) of a mouse after treatment with PBS, C2 compound, and eugenol for 6 days.

Referring to FIG. 21, it was confirmed that in the group treated with PBS, there was no change in body weight of the mouse before and after the experiment, in the group treated with C2, the body weight increased by about 0.5 g, and in the group treated with eugenol, there was a weight loss of about 0.5 g.

In addition, in the feed intake and water intake, it was confirmed that the C2-treated group took similar amounts of feed and water as the PBS group, but the eugenol-treated group took less feed and water.

FIG. 22 is a graph of measuring the weight of lymph nodes in a mouse after treatment with PBS, C2 compound, and eugenol for 6 days.

Referring to FIG. 22, the weight of the lymph nodes in the group treated with the C2 compound was no different from that in the group treated with PBS, but the weight of the group treated with eugenol increased twice or more.

FIG. 23 is a graph of measuring a cell volume (BrdU concentration) and stimulation index of the lymph nodes of a mouse through bromodeoxyuridine assay after treatment with PBS, C2 compound, and eugenol for 6 days, and FIG. 24 is a graph of measuring the amounts of histamine and immunoglobulin E in the blood of the mouse.

Referring to FIG. 23, the number of cells in the auricular lymph nodes extracted from the mouse was measured through BrdU assay, and the stimulation index was calculated based thereon. As a result, it was confirmed that similarly to the weight of the lymph nodes, the group treated with eugenol showed a statistically significant increase in the number of cells and stimulation index of the lymph nodes, but the group treated with C2 compound showed the same cell number and stimulation index as the group treated with PBS.

Referring to FIG. 24, as a result of collecting blood from the mouse and measuring the concentrations of histamine and immunoglobulin E in the blood, it was confirmed that the group treated with the C2 compound showed the histamine concentration similar to that of the PBS group, and rather showed the decreased concentration of immunoglobulin E, whereas the group treated with eugenol showed increased concentrations of both histamine and immunoglobulin E compared to the PBS group.

Through Experimental Example 3, it was confirmed that treatment with the C2 compound not only did not affect the weight loss and feed/water intake of the mouse, but also did not induce the stimulation or in vivo immune response of lymph nodes, and thus had excellent biocompatibility.

### Experimental Example 4. Confirmation of Skin Penetration Efficiency

In order to confirm the skin absorption efficiency of the PDDS series synthesized in Example 1, FIG. 5 showed the results of tracking the positions of oil red O by sectioning the skin tissue at 24 and 48 hours after applying a C2 compound loaded with oil red O as a model drug, and oil red O to the skin of mouse.

Specifically, 100 μL each of oil red O-loaded C2 compound (C2; 50 μL) and oil red O (OR; 100 μL, stock solution: 24 μM in PBS (1% ethanol)) were applied to the skin, and after 24 and 48 hours, the skin tissue was sectioned, and then the fluorescence of oil red O was tracked (excitation: 559 nm, detection: 636±20 nm) using a fluorescence microscope, and quantified and plotted using the Image-J program.

FIG. 25 shows cross-sectional CLSM images (a, d) of mouse skin samples and graphs (b, c, e, f) confirming the absorption rate of oil red O in skin tissue after 24 hours (a to c) and 48 hours (d to f) of treatment with an oil red O only substance and a formulation (C2) in which oil red O was encapsulated into the C2 compound. Here, (b, c) of FIG. 25 and (e, f) of FIG. 25 show the fluorescence intensity plot of Oil red O or the C2 compound (C2) loaded with Oil red O along the yellow dotted line in the images of (a) of FIG. 25 and (d) of FIG. 25, respectively.

Referring to FIG. 25, it was confirmed that the group treated with oil red O alone was hardly absorbed into the subcutaneous tissue even after 24 and 48 hours, but the group treated with the C2 compound loaded with oil red O showed the highest fluorescence intensity in the dermis via the epidermis after 24 hours, and the fluorescence was also measured in the hypodermis.

After 48 hours, it was confirmed that the encapsulated oil red O penetrated deeper and exhibited strong fluorescence in the dermal layer and subcutaneous tissue.

### Experimental Example 5. Confirmation of Anticancer Effect

To confirm the anticancer effect of the PDDS series synthesized in Example 1, the anticancer effect of the C2 compound was evaluated in a melanoma xenograft mouse.

### (1) Anticancer Effect of C2 Compound-Alone Administration

First, in order to analyze the anticancer effect of the C2 compound alone (non-encapsulated drug), B16F10 (mouse murine melanoma cell line) cells were implanted into the left or right thigh (subcutaneously) of a mouse (6-week-old female Balb/c nude) on day 0 to induce a melanoma tumor, and then PBS or the C2 compound was applied topically at 100 μL daily for 16 days, and changes in body weight and tumor volume were measured every 2 to 3 days. In addition, for hemolysis analysis, a blood sample was obtained from the heart of the mouse anesthetized with isoflurane, and then centrifuged at 4° C. (3000 rpm, 3 min) with cold 1×PBS (1 mL, 3 times) to collect red blood cells. The purified red blood cells were treated with 100 μL of 8% (v/v) each of PBS and the C2 compound and further incubated at 37° C. for 1 hour. The mixture was then collected using centrifugation at 4° C. (3000 rpm, 3 min), and the UV/vis absorption peak (492 nm) of the supernatant was measured at 25° C.

Referring to FIG. 26, it was confirmed again that changes in body weight and tumor volume of the mouse treated with the C2 compound alone showed similar results to the mouse treated with PBS, but no toxicity or hemolysis was shown, and thus the biocompatibility of the C2 compound was excellent.

(2) Anticancer Effect of C2 Compound Encapsulated with Anticancer Agent

Next, using the same method as in Experimental Example 2, a C2 compound preparation (encapsulation efficiency=encapsulating rate=14.7%) supported with an anticancer agent (7-ethyl-10-hydroxycamptothecin; SN-38) was prepared and its anticancer effect was confirmed. Specifically, B16F10 (mouse murine melanoma cell line) cells were transplanted into the left or right thigh (subcutaneously) of the mouse (6-week-old female Balb/c nude) on day 0 to induce melanoma tumor, and then after 10 days later (tumor size=about 200 mm³), melanoma model mice were divided into 3 groups (5 mice per group), and each group was administered with 100 μL of PBS, SN-38 (3 mM), or C2+SN-38 (C2 compound encapsulated with SN-38 anticancer agent) by topical application on days 10, 12, 13, and 14 ((a) of FIG. 27). In order to determine toxicity and therapeutic effects in melanoma model mice for 16 days, changes in body weight and tumor volume were measured every 2 to 3 days. At the end of the experiment (day 15), the body of each mouse was photographed with a digital camera to determine the shape and size of the tumor, and at the end of the experiment (day 16), the tumor was extracted from the mouse. The extracted tumor tissue (harvested tumor) was photographed with a digital camera, and the size and volume thereof were measured. In addition, for hemolysis analysis, red blood cells purified from the blood sample obtained from the mouse heart were treated with 100 μL of PBS (negative control), SN-38 (3 mM), C2+SN-38, and Triton-X (0.1% (v/v); positive control) at 8% (v/v), respectively, and further incubated at 37° C. for 1 hour, and then the mixture was centrifuged at 4° C. (3000 rpm, 3 min) and the UV/vis absorption peak (492 nm) of the final obtained supernatant was measured at 25° C.

Referring to (a) to (e) of FIG. 27, there was no significant change in body weight of mice treated with PBS and SN-38 for 15 days, whereas mice treated with C2+SN-38 showed weight gain from day 10, and the mice treated with C2+SN-38 showed a significant decrease in tumor volume during the experimental period (16 days) compared to treatment with PBS and SN-38 alone. As a result of measuring the volume of the extracted tumor tissue, SN-38 only slightly reduced tumor growth, whereas during treatment with C2+SN-38, the tumor size was dramatically reduced, and the average tumor volume of the C2+SN-38 treated group was 281.2±102.1 mm³, while the average tumor volumes of the PBS and SN-38 treated groups were 1845±647.1 mm³ and 1689±498.6 mm³, respectively.

From these results, it was confirmed that the C2 compound (C2+SN-38) encapsulated with the SN-38 anticancer agent may significantly reduce cancer progression and recover melanoma xenograft mice, and it was found that the C2 compound encapsulated with the SN-38 anticancer agent may increase the therapeutic efficacy of the anticancer agent through the skin penetration ability deeper into the skin than that when administered with the anticancer agent alone.

In addition, as a result of analyzing hemolysis to confirm the safety of administration of the C2 compound encapsulated with the SN-38 anticancer agent, as shown in (f) of FIG. 27, severe hemolysis was shown in the case of treatment with SN-38 alone, but C2+SN-38 showed a negligible hemolysis rate of <0.1% in red blood cells. Therefore, it was confirmed that the C2 compound not only reduced the blood toxicity of the encapsulated anticancer agent compared to administration of the anticancer agent alone, but also maintained the high biocompatibility of the C2 compound itself even after encapsulating (loading) the anticancer agent drug.

Hereinabove, the present invention has been described with reference to preferred embodiments thereof. It will be understood to those skilled in the art that the present disclosure may be implemented as modified forms without departing from an essential characteristic of the present disclosure. Therefore, the disclosed embodiments should be considered in an illustrative viewpoint rather than a restrictive viewpoint. The scope of the present invention is defined by the appended claims rather than by the foregoing description, and all differences within the scope of equivalents thereof should be construed as being included in the present invention.

1. A silicone polymer compound represented by Chemical Formula 1 below: 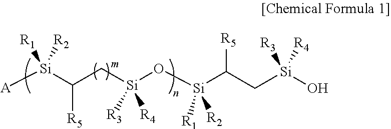
in which,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy,
R₅ is hydrogen or alkyl, and
A is

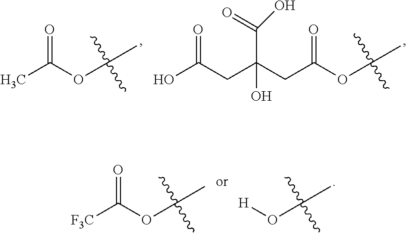

2. The silicone polymer compound of claim 1, wherein the A is 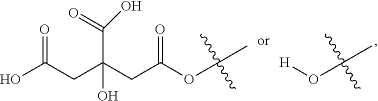
and
the n is an integer of 10 to 35 or 240 to 505.

3. The silicone polymer compound of claim 1, wherein a molecular weight of the silicone polymer compound is 1,000 g/mol to 100,000 g/mol and a viscosity of the polymer compound is 60 cP to 120,000 c.

4. The silicone polymer compound of claim 1, wherein a water contact angle of the polymer compound is 490 or higher.

5. A method for preparing a silicone polymer compound represented by Chemical Formula 1 below, comprising ring-opening polymerizing a monomer represented by Chemical Formula A below using an initiator: 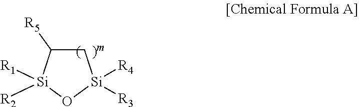
wherein,
m is an integer of 1 to 3,
R₁ to R₄ are each independently any one selected from the group consisting of alkyl, vinyl, acetyl, aromatic, azide, hydroxy, and alkylsiloxy, and
R₅ is hydrogen or alkyl, 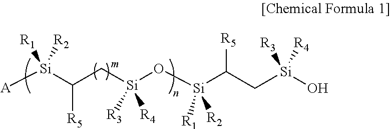
wherein,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy,
R₅ is hydrogen or alkyl, and
A is

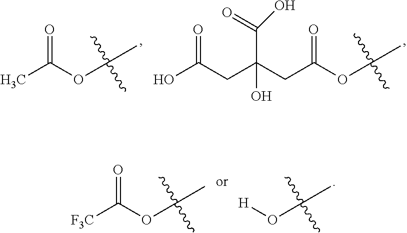

6. The method for preparing the silicone polymer compound of claim 5, wherein the A is 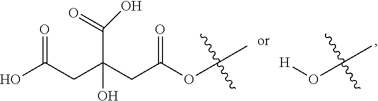
the n is an integer of 10 to 35 or 240 to 505.

7. The method for preparing the silicone polymer compound of claim 5, wherein in the ring-opening polymerization step,
physical properties of the produced silicone polymer compound are controlled by controlling at least any one of a type of initiator, a molar ratio of initiator to monomer, and a polymerization temperature.

8. The method for preparing the silicone polymer compound of claim 7, wherein the controlled physical properties of the controlled silicone polymer compound include at least any one of a molecular weight, a viscosity, a contact angle, and a moisture content.

9. The method for preparing the silicone polymer compound of claim 5, wherein the initiator is at least any one selected from the group consisting of trifluoroacetic acid, phosphoric acid, fumaric acid, acetic acid, sulfonic acid, hydrochloric acid, phosphoric acid, carboxylic acid, sulfonic acid, and citric acid and the initiator is added at a molar ratio of 0.01 to 1.5 based on the monomer.

10. The method for preparing the silicone polymer compound of claim 5, wherein the polymerization step is performed at 10° C. to 100° C. for 10 hours to 100 hours.

11. A composition for transdermal delivery of an active substance wherein the transdermal delivery is delivery to epidermis, dermis, and hypodermis, comprising a silicone polymer compound represented by Chemical Formula 1 below: 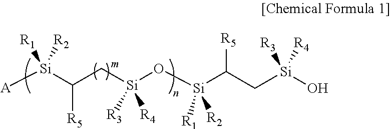
wherein,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy,
R₅ is hydrogen or alkyl, and
A is

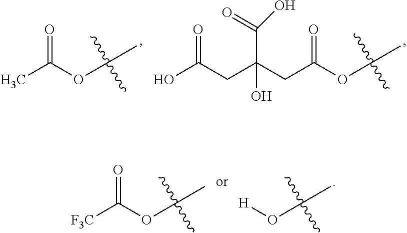

12. The composition for transdermal delivery of claim 11, wherein the A is 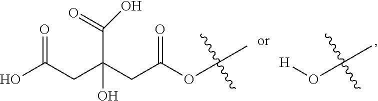
and
the n is an integer of 10 to 35 or 240 to 505.

13. The composition for transdermal delivery of claim 11, wherein in the composition for transdermal delivery, at least any one active substance of a hydrophobic active substance, a hydrophilic active substance, a peptide, and an antibiotic is loaded into the silicone polymer compound.

14. The composition for transdermal delivery of claim 13, wherein the active substance is loaded at an encapsulation ratio of 10% to 99%.

15. A method for transdermal delivery of an active substance, comprising topically applying the composition for transdermal delivery of claim 11 and the active substance, wherein the active substance is at least any one of a hydrophobic active substance, a hydrophilic active substance, a peptide, and an antibiotic.

16. A method for treating cancer comprising administering a composition to a subject in need thereof comprising a silicone polymer compound represented by Chemical Formula 1 below: 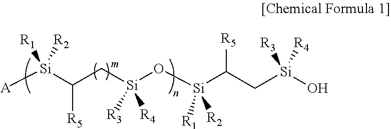
wherein,
m is an integer of 1 to 3,
n is an integer of 10 to 505,
R₁ to R₄ are each independently any one selected from the group consisting of alkyl, vinyl, acetyl, aromatic, azide, hydroxy, and alkylsiloxy,
R₅ is hydrogen or alkyl, and
A is

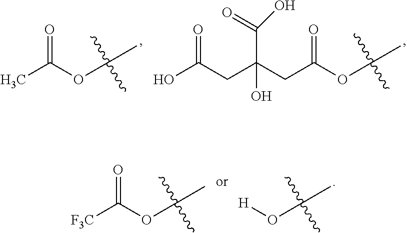

17. The method of claim 16, wherein the A is OH or 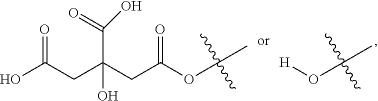
and
the n is an integer of 10 to 35 or 240 to 505.

18. The method of claim 16, wherein the anti-cancer composition further include at least any one anticancer agent selected from the group consisting of SN-38, irinotecan, doxorubicin, paclitaxel, epirubicin, gemcitabine, cilolimus, etoposide, vinblastine, vinca alkaloid, docetaxel, cisplatin, Gleevec, adriamycin, cyclophosphamide, teniposide, 5-fluorouracil, camptothecin, tamoxifen, anastrozole, floxuridine, leuprolide, flutamide, zoledronate, vincristine, streptozotocin, carboplatin, ifosfamide, topotecan, belotecan, irinotecan, vinorelbine, hydroxyurea, valrubicin, methotrexate, mechlorethamine, chlorambucil, busulfan, doxifluridine, prednisone, testosterone, mitoxantrone, docetaxel, vinorelbine, and prednisolone.

19. The method of claim 16, wherein the anticancer agent is loaded into the silicone polymer compound.

20. The method of claim 16, wherein the cancer is any one selected from the group consisting of thyroid cancer, stomach cancer, colon cancer, lung cancer, liver cancer, breast cancer, prostate cancer, gallbladder cancer, biliary tract cancer, pancreas cancer, oral cancer, esophageal cancer, bladder cancer, colon cancer, and cervical cancer.
